(19) United States
(12) Patent Application Publication
(10) Pub. No.: US 2022/0087528 A1
(43) Pub. Date: Mar. 24, 2022

(54) Optical Signal Communication Method and Device, Biometric Data Monitoring System Using the Same

(71) Applicant: Foundation for Research and Business, Seoul National University Science and Technology, Seoul (KR)

(72) Inventor: Ill Soo SOHN, Seoul (KR)

(21) Appl. No.: 17/447,720

(22) Filed: Sep. 15, 2021

(30) Foreign Application Priority Data
Sep. 22, 2020 (KR) 10-2020-0122314
Jun. 16, 2021 (KR) 10-2021-0078185

(51) Int. Cl. A61B 5/00; H04B 10/516

(52) U.S. Cl. CPC A61B 5/0017; A61B 5/0031; H04B 10/516

(57) ABSTRACT

Disclosed are an optical signal communication method and device and a biometric data monitoring system using the optical signal communication method and device. An optical signal communication method performed by an optical signal transmitting device includes receiving current input data to be modulated into an optical signal, dividing the received current input data into a plurality of sub-data sets including first sub-data and second sub-data, modulating the current input data into an optical signal including a start pulse, an intermediate pulse, and an end pulse based on the sub-data sets, and transmitting the optical signal to an optical signal receiving device.

### CROSS-REFERENCE TO RELATED APPLICATIONS

This application claims the benefit of Korean Patent Application No. 10-2020-0122314 filed on Sep. 22, 2020, and Korean Patent Application No. 10-2021-0078185 filed on Jun. 16, 2021, in the Korean Intellectual Property Office, the entire disclosures of which are incorporated herein by reference for all purposes.

### FIELD OF THE DISCLOSURE

One or more example embodiments relate to an optical signal communication technology and a biometric data monitoring technology.

### BACKGROUND

An information and communication technology (ICT)-based medical device has been introduced. Such an ICT-based medical device may be provided in various forms that are attachable to a human body, directly worn on a human body, or implantable in a human body. This medical device may collect various signals and transmit the collected signals to an external device.

An implantable medical device is being more widely used to enhance convenience, stability, and accuracy in collecting biosignals. However, the medical device including an existing radio frequency (RF)-based wireless communication module may consume a great amount of power, and thus use up a battery thereof more rapidly. In addition, the implantable medical device may also have an issue in that an error frequently occurs in a process of transmitting and receiving data. Thus, there is a desire for research to resolve such issues of an implantable medical device.

### SUMMARY

According to an aspect, there is provided an optical signal communication method performed by an optical signal transmitting device, the optical signal communication method including receiving current input data to be modulated into an optical signal, dividing the received current input data into a plurality of sub-data sets including first sub-data and second sub-data, modulating the current input data into an optical signal including a start pulse, an intermediate pulse, and an end pulse based on the sub-data sets, and transmitting the optical signal to an optical signal receiving device. When the optical signal receiving device does not receive at least one of the intermediate pulse and the end pulse from the optical signal transmitting device after the optical signal receiving device receives the start pulse and reaches a time count corresponding to a threshold value, the optical signal receiving device may determine not to demodulate the received optical signal.

The first sub-data may include an upper bit of the current input data, and the second sub-data may include a lower bit of the current input data. A bit number of the first sub-data may be less than a bit number of the second sub-data. An interval between the start pulse and the intermediate pulse may be determined based on a data value of the first sub-data, and an interval between the intermediate pulse and the end pulse may be determined based on a data value of the second sub-data.

According to another aspect, there is provided an optical signal communication method performed by an optical signal transmitting device, the optical signal communication method including receiving current input data to be modulated into an optical signal, modulating the current input data into an optical signal when a difference in value between the current input data and previous input data is greater than a threshold value, and not transmitting the current input data when the difference in value between the current input data and the previous input data is less than or equal to the threshold value, and transmitting, when the difference in value between the current input data and the previous input data is larger than the threshold value, the optical signal to an optical signal receiving device based on a preset transmission period shared between the optical signal transmitting device and the optical signal receiving device.

According to still another aspect, there is provided a biometric data monitoring system including an implantable device configured to be inserted in a body of a target from which biometric data is to be collected, collect the biometric data, modulate the biometric data into an optical signal of a near-infrared light wavelength, and transmit the optical signal to an external optical receiver outside the body, the external optical receiver configured to detect the biometric data from the received optical signal outside the body of the target and transmit the detected biometric data to a remote biometric data collector, and the remote biometric data collector configured to transmit the received biometric data to a server.

Additional aspects of example embodiments will be set forth in part in the description which follows and, in part, will be apparent from the description, or may be learned by practice of the disclosure.

### BRIEF DESCRIPTION OF THE DRAWINGS

These and/or other aspects, features, and advantages of the invention will become apparent and more readily appreciated from the following description of example embodiments, taken in conjunction with the accompanying drawings of which:

FIG. 1 is a diagram illustrating an example of an optical signal communication system according to an example embodiment;

FIG. 2 is a flowchart illustrating an example of an optical signal communication method performed by an optical signal transmitting device according to an example embodiment;

FIG. 3 is a flowchart illustrating another example of an optical signal communication method performed by an optical signal transmitting device according to an example embodiment;

FIG. 4 is a diagram illustrating an example of an optical signal modulated based on divided sub-data according to an example embodiment;

FIG. 5 is a diagram illustrating an example of an optical signal according to an example embodiment;

FIG. 6 is a diagram illustrating an example of an interval in which the same value of a biosignal is maintained according to an example embodiment;

FIG. 7 is a diagram illustrating another example of an optical signal communication method performed among objects according to an example embodiment;

FIGS. 8 and 9 are diagrams illustrating examples of an optical signal according to a first example embodiment;

FIG. 10 is a diagram illustrating an example of an optical signal according to a second example embodiment;

FIG. 11 is a diagram illustrating an example of an optical signal according to a third example embodiment;

FIG. 12 is a diagram illustrating an example of an optical signal transmitting device according to an example embodiment;

FIG. 13 is a diagram illustrating another example of an optical signal transmitting device according to an example embodiment;

FIG. 14 is a diagram illustrating an example of a biometric data monitoring system according to an example embodiment;

FIG. 15 is a flowchart illustrating an example of a biometric data monitoring method performed by an external optical receiver according to an example embodiment;

FIG. 16 is a diagram illustrating an example of a biometric data monitoring method performed among devices included in a biometric data monitoring system according to an example embodiment;

FIGS. 17A and 17B are diagrams illustrating examples of an optical signal according to an example embodiment;

FIG. 18 is a diagram illustrating an example of data to which error detection information and error correction information are added according to the first example embodiment;

FIG. 19 is a diagram illustrating an example of an optical signal in which a start pulse and an end pulse have different widths according to the second example embodiment;

FIG. 20 is a diagram illustrating an example of an optical signal having a threshold value according to the third example embodiment;

FIG. 21 is a diagram illustrating an example of an optical signal in which a start pulse and an end pulse have different wavelengths according to a fourth example embodiment;

FIG. 22 is a diagram illustrating examples of optical signals generated based on sub-biometric data sets obtained by dividing biometric data according to a fifth example embodiment;

FIG. 23 is a diagram illustrating examples of optical signals generated as biometric data is divided into even bits and odd bits according to a sixth example embodiment;

FIG. 24 is a diagram illustrating examples of optical signals each including an identifier bit according to a seventh example embodiment; and

FIG. 25 is a diagram illustrating an example of an external optical receiver according to an example embodiment.

### DETAILED DESCRIPTION

The following structural or functional descriptions of example embodiments described herein are merely intended for the purpose of describing the example embodiments described herein and may be implemented in various forms. However, it should be understood that these example embodiments are not construed as limited to the illustrated forms. Various modifications may be made to the example embodiments. Here, the example embodiments are not construed as limited to the disclosure and should be understood to include all changes, equivalents, and replacements within the idea and the technical scope of the disclosure.

Although terms of “first,” “second,” and the like are used to explain various components, the components are not limited to such terms. These terms are used only to distinguish one component from another component. For example, a first component may be referred to as a second component, or similarly, the second component may be referred to as the first component within the scope of the present disclosure. In addition, when it is mentioned that one component is “connected” or “accessed” to another component, it may be understood that the one component is directly connected or accessed to another component or that still other component is interposed between the two components.

The terminology used herein is for the purpose of describing particular example embodiments only and is not to be limiting of the example embodiments. As used herein, the singular forms “a,” “an,” and “the” are intended to include the plural forms as well, unless the context clearly indicates otherwise. As used herein, the term “and/or” includes any one and any combination of any two or more of the associated listed items. It will be further understood that the terms “comprises” and/or “comprising,” when used in this specification, specify the presence of stated features, integers, steps, operations, elements, components or a combination thereof, but do not preclude the presence or addition of one or more other features, integers, steps, operations, elements, components, and/or groups thereof. In addition, terms such as first, second, A, B, (a), (b), and the like may be used herein to describe components. Each of these terminologies is not used to define an essence, order, or sequence of a corresponding component but used merely to distinguish the corresponding component from other component(s).

Unless otherwise defined herein, all terms used herein including technical or scientific terms have the same meanings as those generally understood by one of ordinary skill in the art. Terms defined in dictionaries generally used should be construed to have meanings matching contextual meanings in the related art and are not to be construed as an ideal or excessively formal meaning unless otherwise defined herein.

Hereinafter, example embodiments will be described in detail with reference to the accompanying drawings. When describing the example embodiments with reference to the accompanying drawings, like reference numerals refer to like components and a repeated description related thereto will be omitted.

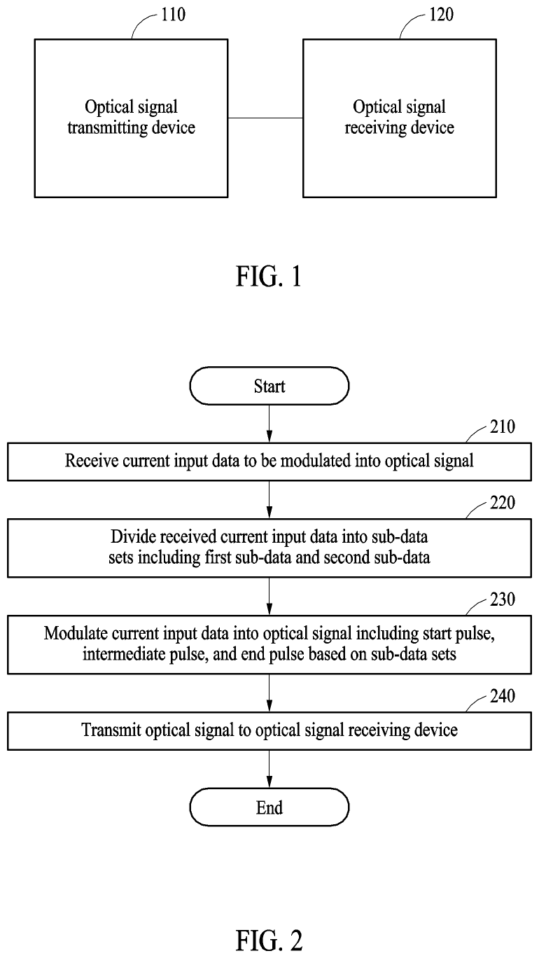

FIG. 1 is a diagram illustrating an example of an optical signal communication system according to an example embodiment.

An optical signal communication system described herein may perform low-power wireless communication using a light-emitting diode (LED) that consumes power only when a power supply is on. The optical signal communication system may divide input data into first sub-data which includes an upper bit of the input data of relatively higher importance, and into second sub-data which includes a lower bit of the input data of relatively lower importance. The optical signal communication system may modulate the input data into an optical signal by determining an interval between a start pulse and an intermediate pulse of the optical signal based on the first sub-data and determining an interval between the intermediate pulse and an end pulse of the optical signal based on the second sub-data. The optical signal communication system may allow a bit number of the first sub-data that is relatively important to be less than a bit number of the second sub-data that is relatively less important, and thereby reduce the probability of potential occurrence of an error during transmission and reception of the relatively important first sub-data and minimize an influence of the error on entire data even when the error occurs.

In addition, when a difference between a value of current input data that is intended to be currently transmitted and a value of previous input data that is previously transmitted is less than a threshold value, the optical signal communication system may skip or omit a process of transmitting the current input data. In such a case, the previous input data may be recognized as the current input data.

Referring to FIG. 1, an optical signal communication method may be performed by an optical signal transmitting device 110 and an optical signal receiving device 120. The optical signal transmitting device 110 may modulate input data into an optical signal and transmit the optical signal to the optical signal receiving device 120. The optical signal receiving device 120 may obtain input data corresponding to the optical signal by demodulating the optical signal received from the optical signal transmitting device 110.

When transmitting the optical signal to the optical signal receiving device 120, the optical signal transmitting device 110 may minimize an amount of time used for a light source to output light and reduce power consumption. In addition, the optical signal transmitting device 110 may divide the input data into a plurality of sub-data sets and modulate the sub-data sets into optical signals each including a start pulse, an intermediate pulse, and an end pulse, and thereby minimize power consumption in a process of transmitting and receiving an optical signal and minimizing an error. During the repetition of the same interval of the input data, the optical signal transmitting device 110 may skip or omit a process of dividing, modulating, and transmitting the input data. When the optical signal receiving device 120 does not receive an optical signal, the optical signal receiving device 120 may determine input data that has not been received based on an optical signal that is most recently received.

The optical signal transmitting device 110 may minimize an error in transmitting an optical signal using at least one of different wavelengths of a start pulse, an intermediate pulse, and an end pulse corresponding to the optical signal, divided input data, different widths of the start pulse, the intermediate pulse, and the end pulse, and error detection information or error correction information included in the optical signal.

Although an example where the optical signal transmitting device 110 divides input data into two sub-data sets is mainly described herein, examples are not limited to the foregoing example. According to examples, the optical signal transmitting device 110 may divide input data into three or more sub-data sets. In addition, the sub-data sets are not limited to being divided by an upper bit and a lower bit, or by even bits and odd bits, but the sub-data sets may be divided in various ways. A sub-data set described herein may indicate a set or piece of sub-data, and sub-data sets described herein may indicate a plurality of sets or pieces of sub-data.

The optical signal receiving device 120 may obtain data corresponding to an optical signal based on a time interval between a start pulse and an intermediate pulse of the optical signal and on a time interval between the intermediate pulse and an end pulse of the optical signal. In addition, the optical signal receiving device 120 may minimize an error in receiving an optical signal using at least one of different wavelengths of a start pulse, an intermediate pulse, and an end pulse corresponding to the optical signal, an identifier bit that indicates a sequence of sub-data sets, different widths of the start pulse, the intermediate pulse, and the end pulse, a threshold value corresponding to a time interval among the start pulse, the intermediate pulse, and the end pulse, and error detection information or error correction information included in the optical signal.

FIG. 2 is a flowchart illustrating an example of an optical signal communication method performed by an optical signal transmitting device according to an example embodiment.

Referring to FIG. 2, in operation 210, an optical signal transmitting device may receive current input data to be modulated into an optical signal. For example, the input data may have a data value in the form of a 16-bit binary. However, the size and form of the input data are not limited to the foregoing example, and the input data may be provided in other sizes and forms.

In operation 220, the optical signal transmitting device may divide the received current input data into a plurality of sub-data sets including first sub-data and second sub-data. The first sub-data may include an upper bit of the current input data, and the second sub-data may include a lower bit of the current input data. In addition, a bit number of the first sub-data may be less than a bit number of the second sub-data, and the bit number of the second sub-data may be greater than the bit number of the first sub-data. In general, data included in an upper bit may be more important than data included in a lower bit. Thus, when an error occurs during transmission of the upper bit, such an error may affect entire data more greatly than when an error occurs during transmission of the lower bit. Based on characteristics of optical signal transmission, a probability of occurrence of an error at a receiving location may increase as an interval between pulses increases. That is, as a bit number to be represented by a single optical signal increases, a probability of occurrence of an error may increase. To minimize an influence of an error on entire data when the error actually occurs, the optical signal transmitting device may perform an asymmetric division that divides, as first sub-data, an upper bit that may more greatly affect the entire data when the error actually occurs than a lower bit and divides, as second sub-data, the lower bit that may less affect the entire data when the error actually occurs than the upper bit. The optical signal transmitting device may thereby minimize the influence of the error even when the error actually occurs.

In operation 230, the optical signal transmitting device may modulate the current input data into an optical signal including a start pulse, an intermediate pulse, and an end pulse based on the sub-data sets obtained by the dividing. An interval between the start pulse and the end pulse may be determined based on a data value of the first sub-data, and an interval between the intermediate pulse and the end pulse may be determined based on a data value of the second sub-data. Since the bit number of the first sub-data is less than the bit number of the second sub-data, the interval between the start pulse and the intermediate pulse may be less than the interval between the intermediate pulse and the end pulse.

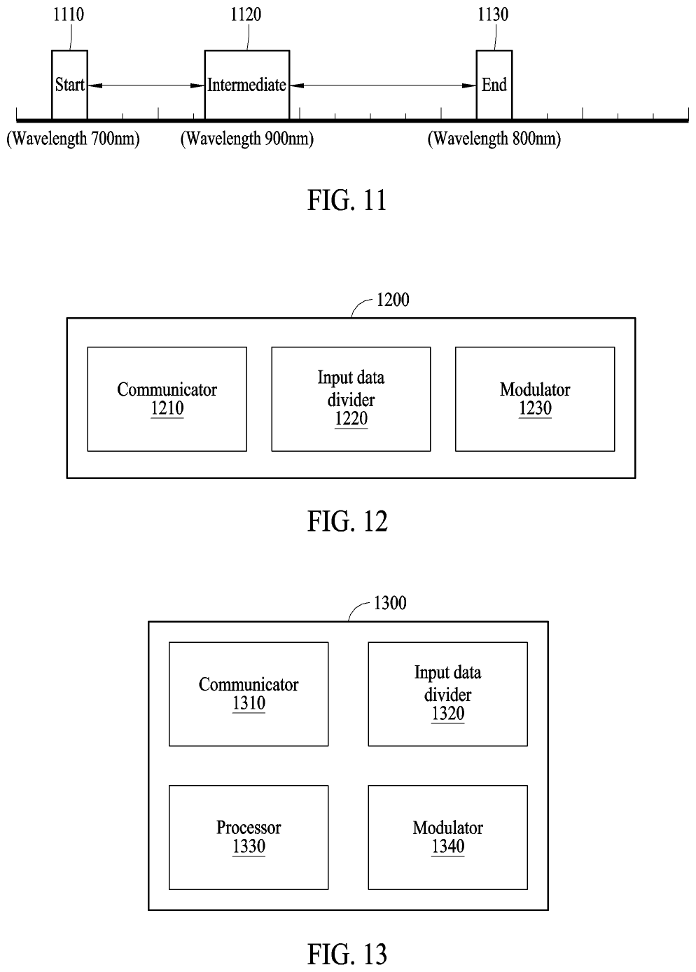

According to another example embodiment, the start pulse, the intermediate pulse, and the end pulse may be identified from each other based on a width or a wavelength of each pulse. That is, the start pulse, the intermediate pulse, and the end pulse may be different in width or wavelength. The identification of the start pulse, the intermediate pulse, and the end pulse by a width or wavelength of each pulse will be described in detail with reference to FIGS. 5 and 11.

According to another example embodiment, the optical signal transmitting device may modulate each of the first sub-data and the second sub-data into an optical signal including a start pulse and an end pulse. That is, the optical signal transmitting device may modulate the first sub-data into a first optical signal including a first start pulse and a first end pulse, and modulate the second sub-data into a second optical signal including a second start pulse and a second end pulse. In this example embodiment, the first end pulse and the second start pulse may be replaced with an intermediate pulse. As an intermediate pulse replaces two pulses as described above, the optical signal transmitting device according to one example embodiment may transmit input data using a smaller number of pulses than an optical signal transmitting device according to another example embodiment.

In operation 240, the optical signal transmitting device may transmit the optical signal to an optical signal receiving device. The optical signal may not include other pulses excluding the start pulse, the intermediate pulse, and the end pulse. When the optical signal receiving device does not receive at least one of the intermediate pulse and the end pulse from the optical signal transmitting device after receiving the start pulse and reaching a time count corresponding to a threshold value, the optical signal receiving device may determine not to demodulate the received optical signal. The threshold value will be described in detail with reference to FIG. 10.

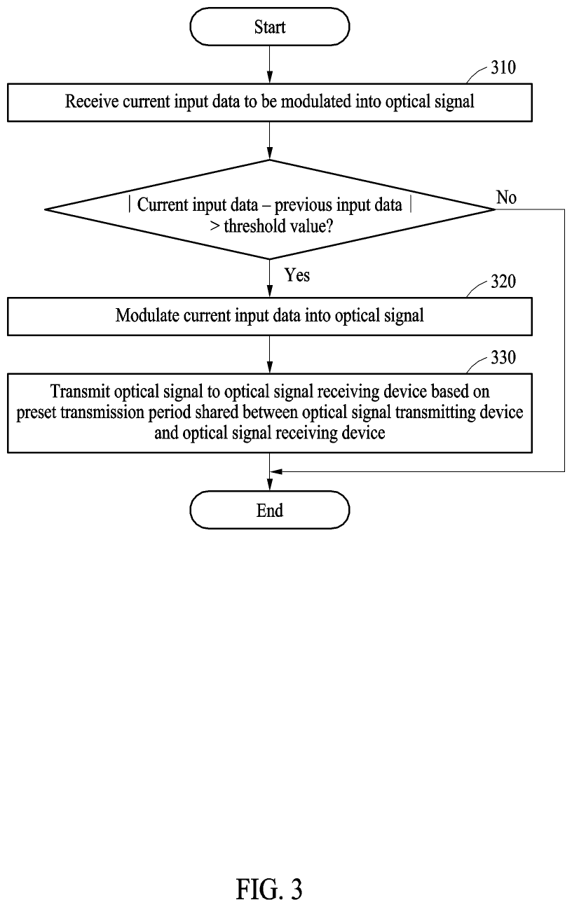

FIG. 3 is a flowchart illustrating another example of an optical signal communication method performed by an optical signal transmitting device according to an example embodiment.

Referring to FIG. 3, in operation 310, an optical signal transmitting device may receive current input data to be modulated into an optical signal. For example, the input data may have a data value in the form of a 16-bit binary. However, the size and form of the input data are not limited to the foregoing example, and the input data may be provided in other sizes and forms.

The optical signal transmitting device may perform a comparison on a difference in value between the current input data and previous input data. When the difference in value between the current input data and the previous input data is less than or equal to a threshold value, the optical signal transmitting device may not transmit the current input data. That is, when the difference between a value of the current input data and a value of the previous input data is less than or equal to the threshold value, the optical signal transmitting device may determine that the value of the current input data and the value of the previous input data are the same, and determine not to transmit the current input data. That is, when the current input data and the previous input data are determined to be the same, the optical signal transmitting device may skip or omit all processes of dividing input data into sub-data sets, modulating the sub-data sets into optical signals, and transmitting the optical signals. Herein, the current input data may be input data that is most recently received, and the previous input data may be input data that is received immediately before the current input data.

In operation 320, when the difference in value between the current input data and the previous input data is greater than the threshold value, the optical signal transmitting device modulate the current input data into an optical signal. For example, the optical signal transmitting device may modulate the current input data into an optical signal including a start pulse, an intermediate pulse, and an end pulse. Alternatively, the optical signal transmitting device may modulate the current input data into an optical signal including at least one of a start pulse, an intermediate pulse, and an end pulse. According to examples, when the difference in value between the current input data and the previous input data is greater than the threshold value, the optical signal transmitting device may divide the current input data into a plurality of sub-data sets. The optical signal transmitting device may modulate the current input data into the optical signal including the start pulse, the intermediate pulse, and the end pulse based on the sub-data sets obtained by the dividing.

In operation 330, the optical signal transmitting device may transmit the optical signal to an optical signal receiving device based on a preset transmission period shared between the optical signal transmitting device and the optical signal receiving device.

The optical signal receiving device may receive a current optical signal from the optical signal transmitting device based on the preset transmission period. According to an example embodiment, the optical signal transmitting device may transmit information associated with the preset transmission period to the optical signal receiving device. Thus, the preset transmission period of the optical signal may be shared in advance between the optical signal transmitting device and the optical signal receiving device. However, in a case in which the optical signal receiving device does not receive the current optical signal from the optical signal transmitting device based on the preset transmission period, the optical signal receiving device may determine the current input data based on a previously received optical signal. When an error occurs in a receiving process, or when the optical signal transmitting device skips or omits transmitting the optical signal corresponding to the current input data based on a determination that the current input data and the previous input data are the same, the optical signal receiving device may not receive the current optical signal based on the preset transmission period. In such a case, the optical signal receiving device may determine, as a current optical signal, a most recently received optical signal among previously received optical signals, and determine the current input data based on the optical signal determined as the current optical signal.

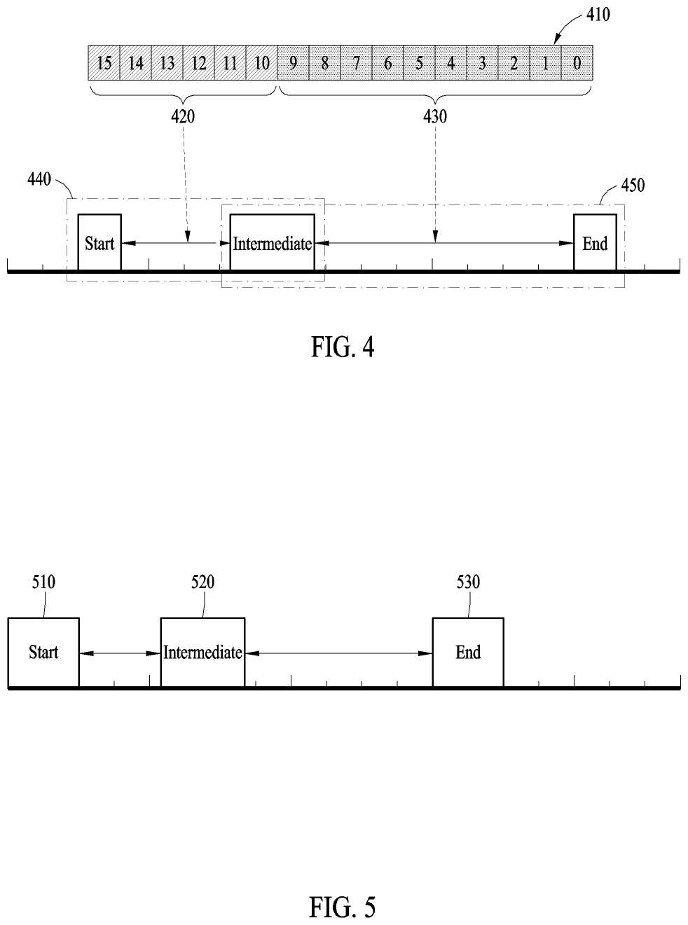

FIG. 4 is a diagram illustrating an example of an optical signal modulated based on divided sub-data according to an example embodiment.

Referring to FIG. 4, input data 410 may be divided into 6-bit first sub-data 420 and 10-bit second sub-data 430. The first sub-data 420 may include an upper bit that may affect entire data more greatly in case of occurrence of an error. That is, the first sub-data 420 may include the upper bit that may more greatly affect the entire data in case of occurrence of an error in transmission and reception, compared to the second sub-data 430. The second sub-data 430 may include a lower bit that may less affect the entire data in case of occurrence of an error in transmission and reception, compared to the first sub-data 420. Since a bit number of the first sub-data 420 is less than a bit number of the second sub-data 430, a probability of an error occurring in a process of transmitting and receiving the first sub-data 420 may be less than a probability of an error occurring in a process of transmitting and receiving the second sub-data 430. Thus, an optical signal transmitting device may minimize an error occurrence probability by modulating data of relatively higher importance into an optical signal based on the first sub-data 420 having a smaller bit number and a relatively less transmission and reception error occurrence probability, and minimize an influence of an error on entire data even when the error occurs.

The optical signal transmitting device may modulate the first sub-data 420 and the second sub-data 430 into respective optical signals each including a start pulse, an intermediate pulse, and an end pulse. An interval 440 between the start pulse and the intermediate pulse may be determined based on the first sub data 420, and an interval 450 between the intermediate pulse and the end pulse may be determined based on the second sub-data 430. An optical signal receiving device may wait for a time corresponding to a first reception threshold value until receiving the intermediate pulse after receiving the start pulse, and wait for a time corresponding to a second reception threshold value until receiving the end pulse after receiving the intermediate pulse.

When the optical signal receiving device does not receive the intermediate pulse or a pulse before the time corresponding to the first reception threshold value elapses after receiving the start pulse, the optical signal receiving device may determine that there is an error in receiving the intermediate pulse and wait to receive the end pulse. In addition, when the optical signal receiving device does not receive the end pulse or a pulse before the time corresponding to the second reception threshold value elapses after receiving the intermediate pulse, the optical signal receiving device may determine that there is an error in receiving the end pulse and wait to receive a start pulse of a subsequent optical signal.

FIG. 5 is a diagram illustrating an example of an optical signal according to an example embodiment.

Referring to FIG. 5, a start pulse 510, an intermediate pulse 520, and an end pulse 530 may have different pulse widths. A pulse width used herein may indicate at least one of a time interval during which a signal value is maintained, a time interval during which a logic level is maintained at 1, and a time interval during which output of light by an optical signal transmitting device is maintained. Since the respective widths of the start pulse 510, the intermediate pulse 520, and the end pulse 530 are different, an optical signal receiving device may identify the start pulse 510, the intermediate pulse 520, and the end pulse 530 from each other based on the widths of the pulses included in an optical signal. When the optical signal receiving device receives the start pulse 510 or the end pulse 530 while not receiving the intermediate pulse 520 after receiving the start pulse 510, the optical signal receiving device may determine that there is an error in receiving the intermediate pulse 520 without misrecognizing the start pulse 510 or the end pulse 530 as the intermediate pulse 520. As described above, the optical signal receiving device may determine that an error occurs in receiving an intermediate pulse without misrecognizing another pulse as the intermediate pulse, and thus prevent the error from affecting an optical signal to be received afterward even when the error occurs.

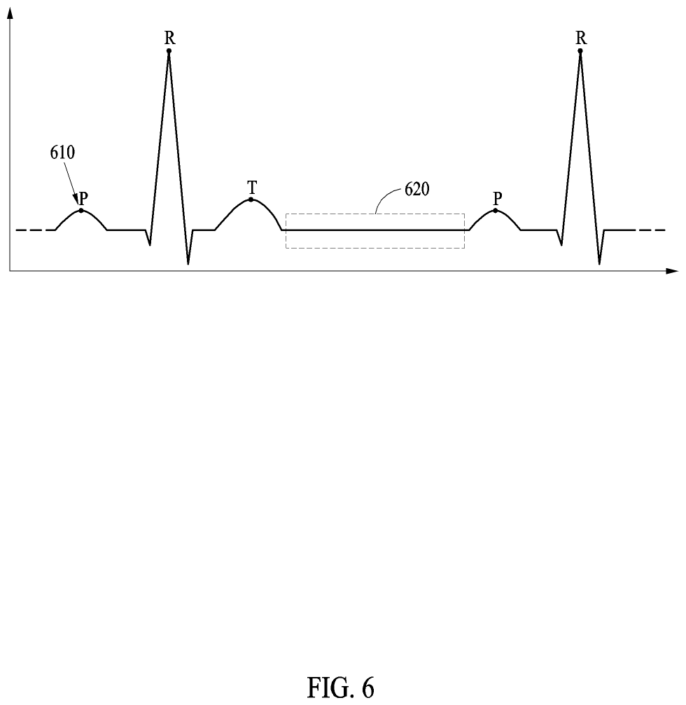

FIG. 6 is a diagram illustrating an example of an interval in which the same value of a biosignal is maintained according to an example embodiment.

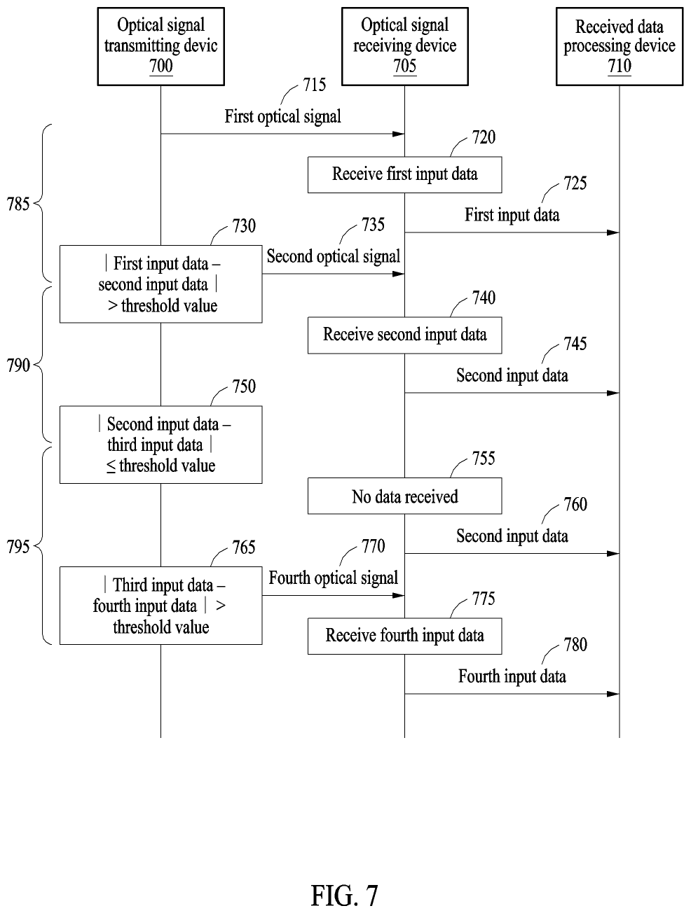

An optical signal communication system described herein may transmit and receive input data 610 that is based on biometric data or biosignals collected by a medical device inserted or implanted in a body. In general, the input data 610 that is based on biometric data may have an interval 620 in which the same value is maintained, and thus the optical signal communication system may skip or omit a process of dividing, modulating, and transmitting the input data 610 for the interval 620 in which the same value of the input data 610 is maintained, in order to minimize energy consumption. For a detailed description of how an optical signal transmitting device skips or omits a process of dividing, modulating, and transmitting input data for an interval in which the same value of the input data is maintained, reference may be made to what is described herein with reference to FIGS. 3 and 7.

FIG. 7 is a diagram illustrating another example of an optical signal communication method performed among objects according to an example embodiment.

Referring to FIG. 7, an optical signal transmitting device 700 may receive first input data from a medical device inserted or implanted in a body of a patient or a user. According to examples, the optical signal transmitting device 700 may be implemented in the body of the user along with the medial device, or be provided outside the body of the user.

The optical signal transmitting device 700 may transmit an optical signal to an optical signal receiving device 705 based on a preset transmission period, for example, a transmission period 785, a transmission period 790, or a transmission period 795. Thus, the optical signal receiving device 705 may receive an optical signal from the optical signal transmitting device 700 based on the preset transmission period 785, 790, or 795. For example, the optical signal transmitting device 700 may transmit a first optical signal to the optical signal receiving device 705, and then transmit a second optical signal to the optical signal receiving device 705 after an amount of time corresponding to the preset transmission period 785 elapses after transmitting the first optical signal.

When the optical signal receiving device 705 does not receive the optical signal from the optical signal transmitting device 700 based on the transmission period 785, 790, or 795, the optical signal receiving device 705 may determine that there is an error occurring in a process of transmitting and receiving the optical signal or that current input data and previous input data are determined to be the same.

The transmission periods 785, 790, and 795 may correspond to the same time interval. The transmission periods 785, 790, and 795 may be determined in advance and shared between the optical signal transmitting device 700 and the optical signal receiving device 705. For example, the optical signal transmitting device 700 may transmit a preset transmission period to the optical signal receiving device 705 to share the transmission period with the optical signal receiving device 705.

The optical signal transmitting device 700 may divide the first input data into a plurality of sub-data sets, and modulate the sub-data sets into the first optical signal. In operation 715, the optical signal transmitting device 700 may transmit the first optical signal to the optical signal receiving device 705. In operation 720, the optical signal receiving device 705 may receive the first input data based on the first optical signal. That is, the optical signal receiving device 705 may determine the first input data based on the first optical signal. In operation 725, the optical signal receiving device 705 may transmit the first input data to a received data processing device 710.

The optical signal transmitting device 700 may receive second input data. In operation 730, the optical signal transmitting device 700 may calculate whether a difference in value between the first input data and the second input data is greater than a threshold value. In operation 735, when the difference in value between the first input data and the second input data is greater than the threshold value, the optical signal transmitting device 700 may transmit the second optical signal that is based on the second input data to the optical signal receiving device 705. In operation 740, the optical signal receiving device 705 may receive the second input data based on the second optical signal. In operation 745, the optical signal receiving device 705 may transmit the second input data to the received data processing device 710.

The optical signal transmitting device 700 may receive third input data. In operation 750, the optical signal transmitting device 700 may calculate whether a difference in value between the second input data and the third input data is greater than a threshold value. When the difference in value between the second input data and the third input data is less than or equal to the threshold value, the optical signal transmitting device 700 may skip or omit a process of dividing and modulating the third input data, and skip or omit a process of transmitting a third optical signal. In operation 755, the optical signal receiving device 705 may recognize that there is no data received based on the preset transmission period 790. In operation 760, the optical signal receiving device 705 may recognize that the third input data and the second input data are determined to be the same, and transmit the second input data as the third input data to the received data processing device 710.

The optical signal transmitting device 700 may receive fourth input data. In operation 765, the optical signal transmitting device 700 may calculate whether a difference in value between the third input data and the fourth input data is greater than a threshold value. In operation 770, when the difference in value between the third input data and the fourth input data is greater than the threshold value, the optical signal transmitting device 700 may transmit a fourth optical signal that is based on the fourth input data to the optical signal receiving device 705. In operation 775, the optical signal receiving device 705 may receive the fourth input data based on the fourth optical signal. In operation 780, the optical signal receiving device 705 may transmit the fourth input data to the received data processing device 710.

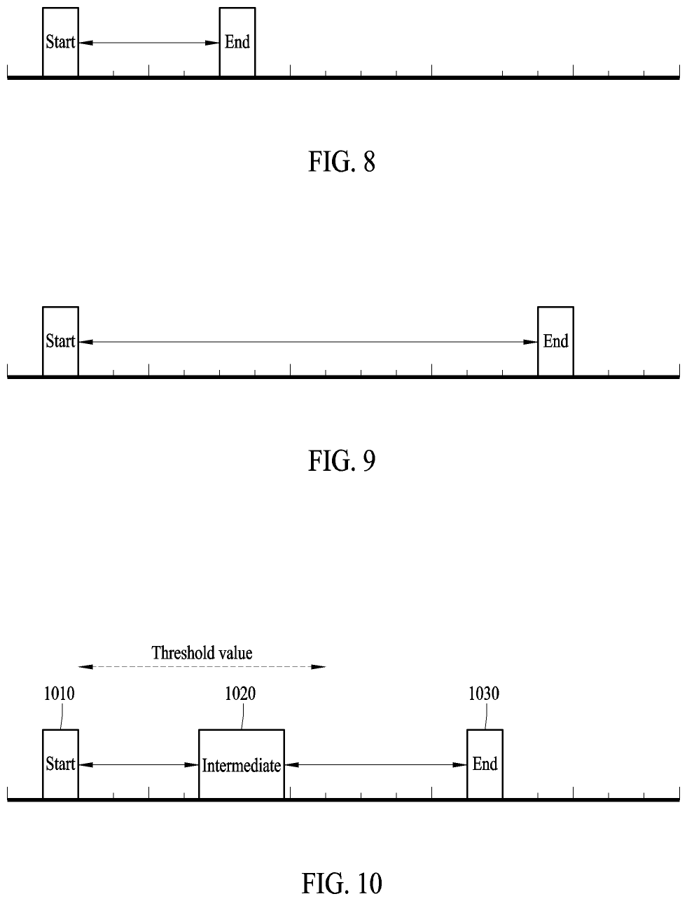

FIGS. 8 and 9 are diagrams illustrating examples of an optical signal according to a first example embodiment.

Referring to FIGS. 8 and 9, an optical signal may include a start pulse and an end pulse. An optical signal transmitting device may determine a time interval between a time point at which the start pulse is transmitted and a time point at which the end pulse is transmitted, based on a data value of input data. Here, the optical signal transmitted by the optical signal transmitting device may include only the start pulse and the end pulse. In addition, the optical signal transmitting device may minimize power consumption by outputting light only when transmitting the start pulse and when transmitting the end pulse. An optical signal receiving device may determine data corresponding to the optical signal based on a time interval between a time point at which the start pulse is received and a time point at which the end pulse is received.

Referring to FIG. 8, according to the first example embodiment, the time interval between the time point at which the optical signal transmitting device transmits the start pulse or the optical signal receiving device receives the start pulse, and the time point at which the optical signal transmitting device transmits the end pulse or the optical signal receiving device receives the end pulse may correspond to four spaces. Each of the spaces present between the start pulse and the end pulse may indicate a preset time interval. In this case, the data value of the data corresponding to the optical signal may be 4, and the optical signal receiving device may determine the data value as 4 based on the time interval between the time points at which the start pulse and the end pulse are received. According to examples, the optical signal receiving device may demodulate and process the data value into an 8-bit binary.

Referring to FIG. 9, according to the first example embodiment, the time interval between the time point at which the optical signal transmitting device transmits the start pulse or the optical signal receiving device receives the start pulse, and the time point at which the optical signal transmitting device transmits the end pulse or the optical signal receiving device receives the end pulse may correspond to 13 spaces. In this case, the data value of the data corresponding to the optical signal may be 13, and the optical signal receiving device may determine the data value as 13 based on the time interval between the time points at which the start pulse and the end pulse are received.

FIG. 10 is a diagram illustrating an example of an optical signal according to a second example embodiment.

FIG. 10 is provided to illustrate an optical signal having a threshold value according to the second example embodiment.

Referring to FIG. 10, according to the second example embodiment, an optical signal transmitting device may set a threshold value for an optical signal and share the set threshold value with an optical signal receiving device. Here, a threshold value for an optical signal may be a maximum value of a time interval between pulses corresponding to the (single) optical signal. For example, as illustrated, a maximum value of a time interval between a start pulse 1010 and an intermediate pulse 1020 may be a first threshold value, and a maximum value of a time interval between the intermediate pulse 1020 and an end pulse 1030 may be a second threshold value. According to examples, the first threshold value and the second threshold value may be the same value, or the first threshold value may be less than the second threshold value.

Referring to FIG. 10, the optical signal transmitting device may determine a threshold value including the first threshold value and the second threshold value, and share the determined threshold value with the optical signal receiving device. The optical signal receiving device may receive the start pulse 1010 corresponding to a first optical signal, and then receive the intermediate pulse 1020 corresponding to the first optical signal. The optical signal receiving device may receive normally first sub-data of the first optical signal by receiving the intermediate pulse 1020 corresponding to the first optical signal before reaching a time count corresponding to the first threshold value after receiving the start pulse 1010 corresponding to the first optical signal. In addition, the optical signal receiving device may receive normally second sub-data of the first optical signal by receiving the end pulse 1030 corresponding to the first optical signal before reaching a time count corresponding to the second threshold value after receiving the intermediate pulse 1020.

The optical signal receiving device may receive a start pulse corresponding to a second optical signal after receiving the end pulse 1030 corresponding to the first optical signal. The optical signal receiving device may wait to receive an intermediate pulse and an end pulse corresponding to the second optical signal to receive normally the second optical signal.

When an error occurs in receiving one pulse in a case in which the optical signal receiving device does not identify the start pulse 1010, the intermediate pulse 1020, and the end pulse 1030 from each other using only a pulse width because the start pulse 1010, the intermediate pulse 1020, and the end pulse 1030 have the same width, the error may affect the reception of a subsequent optical signal by the optical signal receiving device. In such a case, in the absence of a threshold value, when the optical signal receiving device does not receive the intermediate pulse 1020 corresponding to the first optical signal after receiving the start pulse 1010 corresponding to the first optical signal, the optical signal receiving device may extract erroneous data from the first optical signal and such an error may affect the identification of an optical signal to be received afterward.

However, according to the second example embodiment, in the presence of a threshold value, when the optical signal receiving device does not receive the intermediate pulse 1020 corresponding to the first optical signal even after receiving the start pulse 1010 corresponding to the first optical signal, starting a time count corresponding to the first threshold value, and then reaching the time count, the optical signal receiving device may determine that an error occurs in receiving the first sub-data of the first optical signal based on the first threshold value. In addition, when the optical signal receiving device does not receive the end pulse 1030 corresponding to the first optical signal even after receiving the intermediate pulse 1020 corresponding to the first optical signal, starting a time count corresponding to the second threshold value, and then reaching the time count, the optical signal receiving device may determine that an error occurs in receiving the second sub-data of the first optical signal based on the second threshold value. When it is determined that there is an error, the optical signal receiving device may determine not to demodulate an optical signal corresponding to a pulse that is not received.

After the optical signal receiving device receives the start pulse 1010 corresponding to the first optical signal, a time count may start. In such a case, when the optical signal receiving device does not receive the intermediate pulse 1020 corresponding to the first optical signal within the time count corresponding to the first threshold value, the optical signal receiving device may suspend the time count corresponding to the first threshold value, and start a time count corresponding to the second threshold value while waiting for the end pulse 1030. Alternatively, the optical signal receiving device may determine not to demodulate the optical signal corresponding to the start pulse 1010.

FIG. 11 is a diagram illustrating an example of an optical signal according to a third example embodiment.

FIG. 11 is provided to illustrate an optical signal in which a start pulse, an intermediate pulse, and an end pulse have different wavelengths.

Referring to FIG. 11, according to the third example embodiment, an optical signal may include a start pulse 1110, an intermediate pulse 1120, and an end pulse 1130. The start pulse 1110, the intermediate pulse 1120, and the end pulse 1130 may have different wavelengths.

According to this example embodiment, an optical signal transmitting device may transmit, to an optical signal receiving device, the start pulse 1110, the intermediate pulse 1120, and the end pulse 1130 corresponding to the optical signal. The optical signal receiving device may wait until receiving the intermediate pulse 1120 after receiving the start pulse 1110 corresponding to the optical signal. The optical signal receiving device may receive the intermediate pulse 1120 corresponding to the optical signal, and start a time count until receiving the end pulse 1130 in response to the reception of the intermediate pulse 1120.

The respective wavelengths of the start pulse 1110, the intermediate pulse 1120, and the end pulse 1130 may be different from each other. For example, the optical signal transmitting device may set the wavelength of the start pulse 1110 to be 700 nm, the wavelength of the intermediate pulse 1120 to be 900 nm, and the wavelength of the end pulse 1130 to be 800 nm. The optical signal receiving device may identify the start pulse 1110, the intermediate pulse 1120, and the end pulse 1130 from one another based on a characteristic that their wavelengths are different, and thereby recognize and distinguish the start pulse 1110, the intermediate pulse 1120, and the end pulse 1130.

Based on the characteristic that the wavelengths of the start pulse 1110, the intermediate pulse 1120, and the end pulse 1130 are different, the optical signal receiving device may recognize the start of reception of the optical signal when the optical signal receiving device receives the start pulse 1110 corresponding to the optical signal. The optical signal receiving device recognizing the start of the reception of the optical signal may start a time count. When the optical signal receiving device receives the end pulse 1130 while waiting to receive the intermediate pulse 1120 corresponding to the optical signal, the optical signal receiving device may determine that an error occurs in receiving the intermediate pulse 1120.

The optical signal receiving device may identify the start pulse 1110, the intermediate pulse 1120, and the end pulse 1130 based on the wavelengths of the start pulse 1110, the intermediate pulse 1120, and the end pulse 1130. Thus, even when an error occurs in a process of transmitting and receiving an optical signal, it is possible to minimize an influence of the error on the processing of an optical signal to be subsequently received by the optical signal receiving device.

FIG. 12 is a diagram illustrating an example of an optical signal transmitting device according to an example embodiment.

Referring to FIG. 12, an optical signal transmitting device 1200 may include a communicator 1210, an input data divider 1220, and a modulator 1230. The optical signal transmitting device 1200 described herein with reference to FIG. 12 may correspond to the optical signal transmitting device described above with reference to FIG. 2.

The communicator 1210 may receive current input data to be modulated into an optical signal, and transmit the optical signal to an optical signal receiving device.

The input data divider 1220 may divide the received current input data into a plurality of sub-data sets including first sub-data and second sub-data. The modulator 1230 may modulate the current input data into an optical signal including a start pulse, an intermediate pulse, and an end pulse, based on the sub-data sets obtained by the dividing.

FIG. 13 is a diagram illustrating another example of an optical signal transmitting device according to an example embodiment.

Referring to FIG. 13, an optical signal transmitting device 1300 may include a communicator 1310, an input data divider 1320, a processor 1330, and a modulator 1340. The optical signal transmitting device 1300 described herein with reference to FIG. 13 may correspond to the optical signal transmitting device described above with reference to FIG. 3.

The communicator 1310 may receive current input data to be modulated into an optical signal, and transmit the optical signal to an optical signal receiving device. The processor 1330 may calculate whether a difference in value between the current input data and previous input data is greater than a threshold value. When the difference in value between the current input data and the previous input data is less than or equal to the threshold value, the processor 1330 may determine not to transmit the current input data.

When the difference in value between the current input data and the previous input data is greater than the threshold value, the input data divider 1320 may divide the current input data into a plurality of sub-data sets.

The modulator 1340 may modulate the sub-data sets into an optical signal including a plurality of pulses. When the difference in value between the current input data and the previous input data is greater than the threshold value, the modulator 1340 may modulate the current input data into an optical signal including a start pulse, an intermediate pulse, and an end pulse, based on the sub-data sets obtained by the dividing.

According to an example embodiment, functions and operations of the input data divider 1320 and the modulator 1340 may be performed by one or more processors.

According to examples, the optical signal transmitting device 1200 of FIG. 12 may perform the optical signal communication method described above with reference to FIG. 3, and the optical signal transmitting device 1300 of FIG. 13 may perform the optical signal communication method described above with reference to FIG. 2.

According to an example embodiment, it is possible to develop an ultra-low power wireless data transmission communication module for an implantable medical biometric monitoring device.

According to example embodiments described herein, indicating information at intervals among a start pulse, an intermediate, and an end pulse may reduce an amount of time used for a light source to output light and reduce power consumption.

According to example embodiments described herein, it is possible to prevent an error such as omission of data that may occur in a wireless data transmission and reception process, and minimize an influence of the error on data to be transmitted and received afterward even when the error occurs.

According to example embodiments described herein, by dividing an upper bit of data which is of relatively higher importance as first sub-data having a less bit number than second sub-data and dividing a lower bit of data as the second sub-data having a greater bit number than the first sub-data, it is possible to improve the accuracy in data transmission and reception.

According to example embodiments described herein, setting a maximum value of a time interval for receiving an end pulse after receiving a start pulse may minimize an influence of an error on a signal to be transmitted and received afterward even when the error occurs during signal transmission and reception.

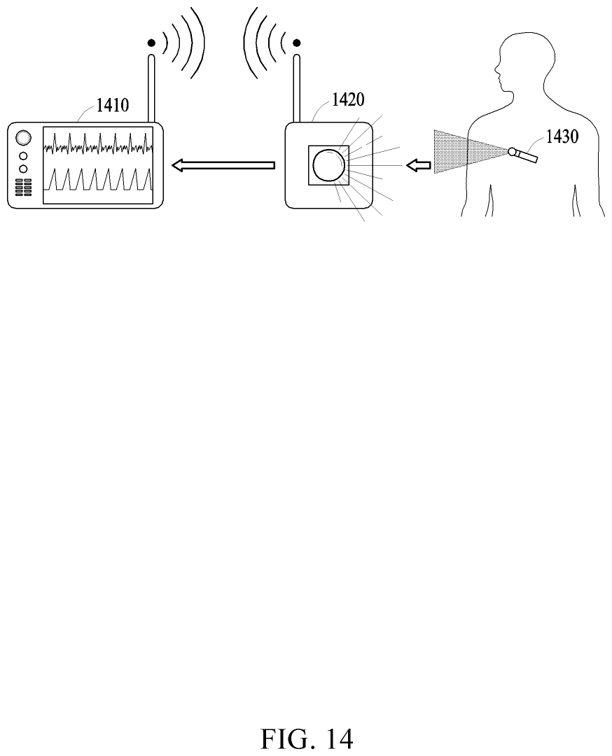

FIG. 14 is a diagram illustrating an example of a biometric data monitoring system according to an example embodiment.

A biometric data monitoring system described herein may collect, in real time, biometric data through a device inserted or implanted in a body of a target (or a user or patient) from which biometric data is to be collected based on an optical communication method harmless to a human body, and monitor the biometric data. When an abnormal symptom occurs, the biometric data monitoring system may transmit related data for notifying a state of the target to a terminal of a related institution or facility, a family, or an acquaintance.

Referring to FIG. 14, a biometric data monitoring system may include an implantable device 1430 that is inserted or implanted inside a body, an external optical receiver 1420, and a remote biometric data collector 1410. According to an example embodiment, the implantable device 1430 may correspond to an optical signal transmitting device described herein, for example, the optical signal transmitting device 110 of FIG. 1, and the external optical receiver 1420 may correspond to an optical signal receiving device described herein, for example, the optical signal receiving device 120 of FIG. 1. The implantable device 1430 may perform all the functions and operations of the optical signal transmitting device described above, and the external optical receiver 1420 may perform all the functions and operations of the optical signal receiving device described above.

For example, the implantable device 1430 may receive current input data to be modulated into an optical signal, divide the received current input data into a plurality of sub-data sets including first sub-data and second sub-data, modulate the current input data into an optical signal including a start pulse, an intermediate pulse, and an end pulse based on the sub-data sets, and transmit the optical signal to the external optical receiver 1420. When at least one of the intermediate pulse and the end pulse is not received from the implantable device 1430 after the start pulse is received and a time count corresponding to a threshold value starts, the external optical receiver 1420 may determine not to demodulate the received optical signal.

According to an example embodiment, the implantable device 1430 may collect biometric data as being inserted in a body of a target from which the biometric data is to be collected, modulate the biometric data into an optical signal of a near-infrared light wavelength, and transmit the optical signal to the external optical receiver 1420. Here, the wavelength of the optical signal may have a value between 600 nanometers (nm) and 1000 nm in skin-permeable wavelength. The implantable device 1430 may include a battery, a biometric data measurer, a biometric data processor, and a communicator.

Outside the body of the target, the external optical receiver 1420 may detect the biometric data from the received optical signal and transmit the detected biometric data to the remote biometric data collector 1410. When a preset event is detected, the external optical receiver 1420 may determine that an abnormal symptom occurs in the target and record an event detection time at which the event is detected. The external optical receiver 1420 may include a battery, an optical communicator, a biometric data processor, an abnormal symptom detection event recording button, and a radio frequency (RF) communicator.

The remote biometric data collector 1410 may transmit the received biometric data to a server. The remote biometric data collector 1410 may transmit, to the server, at least one of the received biometric information and information associated with the event detection time. The remote biometric data collector 1410 may include a battery, a biometric data processor, an alarm signal generator, an abnormal symptom detection event recording button, a biometric data storage, and an RF communicator.

When the information associated with the event detection time is received from the remote biometric data collector 1410, the server may transmit, to a terminal of a preset recipient address, alarm information notifying that the abnormal symptom occurs in the target.

Hereinafter, a biometric data monitoring method will be described in detail.

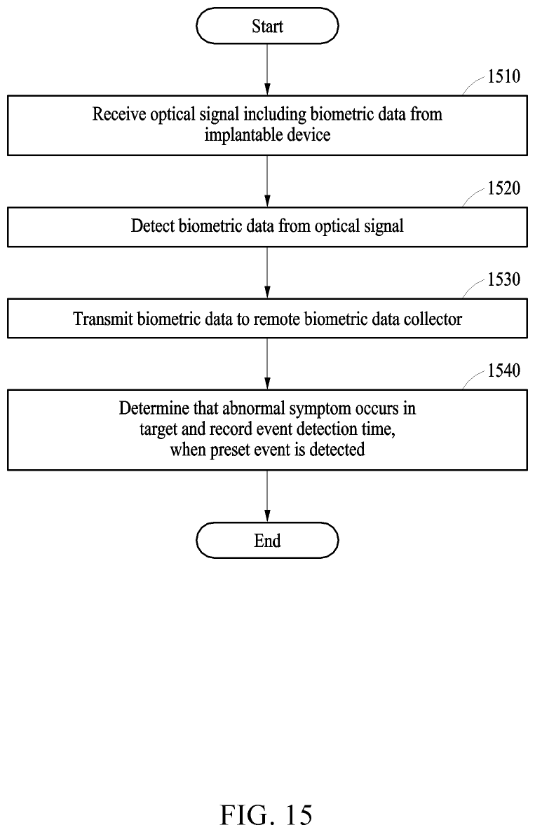

FIG. 15 is a flowchart illustrating an example of a biometric data monitoring method performed by an external optical receiver according to an example embodiment.

Referring to FIG. 15, in operation 1510, an external optical receiver may receive an optical signal including biometric data from an implantable device.

The implantable device may collect the biometric data through an in-body medical device, such as, for example, an electrocardiogram (ECG) measurer, a capsule endoscope, and a blood sugar measurer, as being inserted or implanted in a body of a target from which the biometric data is to be collected. The implantable device may modulate the biometric data into an optical signal of a near-infrared light wavelength and transmit the optical signal to an external optical receiver. The external optical receiver may receive the optical signal transmitted from the implantable device.

According to an example embodiment, the implantable device may modulate the biometric data into an optical signal including a start pulse and an end pulse. According to another example embodiment, the implantable device may divide the biometric data into first sub-biometric data and second sub-biometric data, and modulate the first sub-biometric data and the second sub-biometric data into a first optical signal and a second optical signal, respectively. Each of the first optical signal and the second optical signal may include a start pulse and an end pulse. When an amount of biometric data is large, the implantable device may divide the biometric data into two or more sub-biometric data sets to improve the accuracy in optical signal communication. Communication between the implantable device and the external optical receiver may be based on an ultra-low power optical pulse transmission technology, and use a near-infrared light wavelength (wavelength: 600 to 1000 nm) that is permeable to skin. Here, an optical signal may be harmless to a human body, and thus the external optical receiver and the implantable device may transmit and receive the optical signal without causing harm to a target from which biometric data is to be collected.

According to another example embodiment, when the external optical receiver does not receive the end pulse before a time count corresponding to a threshold value is completed after receiving the start pulse, the external optical receiver may determine that an error occurs in receiving an optical signal corresponding to the start pulse and determine not to demodulate the optical signal. The threshold value may be a maximum value of a time interval between a time point at which the start pulse is recognized and a time point at which the end pulse is recognized. A biometric data monitoring system may set the threshold value for a time until the external optical receiver receives the end pulse after the external optical receiver receives the start pulse. Thus, even when an error that the end pulse is not received after the start pulse is received occurs, the biometric data monitoring system may prevent the error from affecting signals to be received afterward.

In operation 1520, the external optical receiver may detect the biometric data from the received optical signal, outside the body of the target.

The optical signal may include the start pulse and the end pulse having different widths. The start pulse and the end pulse have different widths, and thus the external optical receiver may identify the start pulse and the end pulse from each other.

The optical signal may include only the start pulse and the end pulse, without other pulses. Thus, it may be effective in low-power transmission and reception of the optical signal by the implantable device and the external optical receiver, and in extending the life of a battery.

A time interval between the start pulse and the end pulse may be determined based on the biometric data, and thus the external optical receiver may detect the biometric data from the optical signal based on the time interval between the start pulse and the end pulse included in the optical signal.

In operation 1530, the external optical receiver may transmit the biometric data to a remote biometric data collector. The remote biometric data collector may transmit the received biometric data to a server (or cloud). Transmission and reception of the biometric data performed between the external optical receiver and the remote biometric data collector may be based on RF communication, such as, for example, Bluetooth, WiFi, and cellular communication. The communication between the external optical receiver and the remote biometric data collector may be performed outside a human body, and thus less influential to the harmfulness to a human body. Thus, using an RF communication method that is effective in omnidirectional transmission, it is possible to facilitate the arrangement of the remote biometric data collector and improve the mobility of a user.

In operation 1540, when a preset event is detected, the external optical receiver may determine that an abnormal symptom occurs in the target and record an event detection time at which the event is detected. Here, the preset event may be detected, for example, in a case where the external optical receiver receives a user input to a preset button. The user input to the preset button may be received, for example, when a user inputs a button of the external optical receiver provided in the form of a necklace, or the form of a skin-attachable or clothes-attachable patch.

In addition, the preset event may be detected, for example, in at least one of a case where a preset application that detects an abnormal symptom of the target is executed in the remote biometric data collector, and a case where a preset operation is detected in the remote biometric data collector. The case where the preset operation is detected in the remote biometric data collector may include a case where the user shakes the remote biometric data collector at a certain or higher intensity, or the user activates a preset switch of the remote biometric data collector. That is, the preset event is detected in at least one of cases where a preset switch of the remote biometric data collector is activated, and where the remote biometric data collector is shaked at a certain or higher intensity.

The remote biometric data collector may transmit, to the server, at least one of the received biometric data and the information associated with the event detection time. Communication between the remote biometric data collector and the server may be performed through wired or wireless Internet.

The remote biometric data collector may adjust an information amount of the biometric data to be transmitted based on at least one of a degree of importance and urgency of transmission of biometric data, a communication amount (or traffic) required for transmission, and a residual battery amount. The remote biometric data collector may transmit all the collected biometric data, compress all the biometric data and transmit the compressed data, or transmit only biometric data corresponding to the information associated with the event detection time, based on at least one of a numerical value of a degree of severity of the abnormal symptom, a communication amount (or traffic) required for transmission of the biometric data, or a residual battery amount. For example, when the residual battery amount is low, failing to satisfy a preset reference, the remote biometric data collector may transmit, to the server, only the biometric data corresponding to the time information recorded by the abnormal symptom in order to minimize a battery usage amount. For another example, when the communication amount required for the transmission is greater than a preset amount, the remote biometric data collector may compress all the collected biometric data and transmit the compressed data to the server, or transmit only the biometric data corresponding to the time information recorded by the abnormal symptom to the server. The remote biometric data collector may be, for example, a user terminal in which an application of monitoring the biometric data is executed, or a dedicated stationary or mobile receiver.

When the information associated with the event detection time is received from the remote biometric data collector, the server may transmit, to a terminal of a preset recipient address, alarm information notifying that the abnormal symptom occurs in the target. The preset recipient address may include at least one of a hospital/doctor, a public office/local government office, a family/acquaintance, or the target himself/herself.

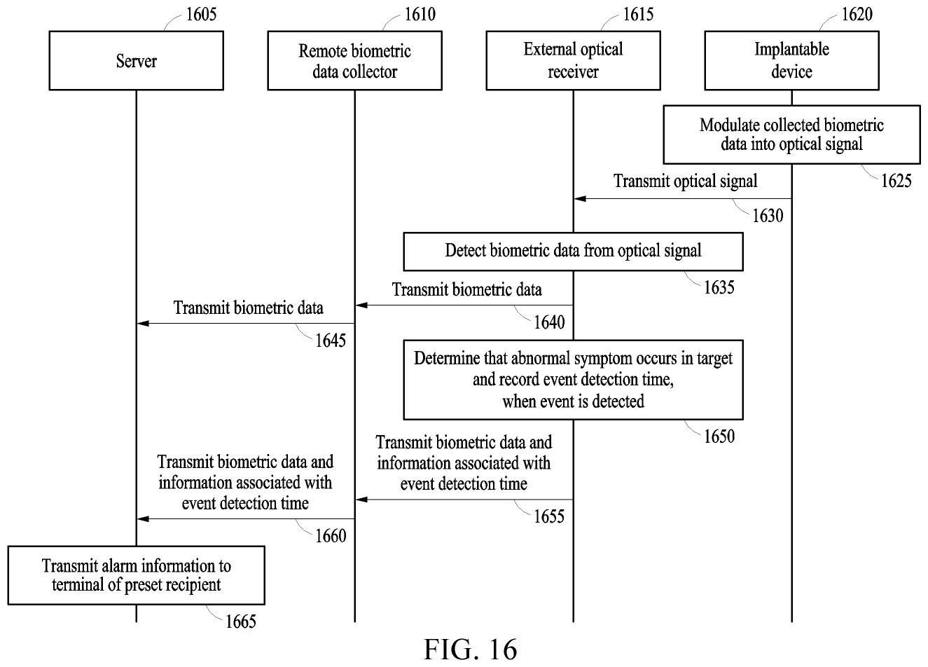

FIG. 16 is a diagram illustrating an example of a biometric data monitoring method performed among devices included in a biometric data monitoring system according to an example embodiment.

Referring to FIG. 16, a biometric data monitoring system may include an implantable device 1620, an external optical receiver 1615, a remote biometric data collector 1610, and a server 1605.

The implantable device 1620 may collect biometric data, as being inserted or implanted in a body of a target from which the biometric data is to be collected. In operation 1625, the implantable device 1620 may modulate the collected biometric data into an optical signal of a near-infrared light wavelength. The implantable device 1620 may modulate the biometric data into an optical signal including only a start pulse and an end pulse. According to examples, the implantable device 1620 may divide the biometric data into two or more sub-biometric data sets, and modulate each of the sub-biometric data sets into an optical signal including only a start pulse and an end pulse.

In operation 1630, the implantable device 1620 may transmit the optical signal to the external optical receiver 1615. The implantable device 1620 may transmit the optical signal by transmitting the start pulse to the external optical receiver 1615 and then transmitting the end pulse to the external optical receiver 1615.

In operation 1635, the external optical receiver 1615 may detect the biometric data from the received optical signa. An interval between the start pulse and the end pulse included in the optical signal may be determined based on the biometric data, and thus the external optical receiver 1615 may detect the biometric data from the optical signal based on the start pulse and the end pulse included in the optical signal.

In operation 1640, the external optical receiver 1615 may transmit the biometric data detected from the optical signal to the remote biometric data collector 1610. In operation 1645, the remote biometric data collector 1610 may transmit the biometric data to the server 1605.

In operation 1650, when a preset event is detected, the external optical receiver 1615 may determine that an abnormal symptom occurs in the target and record an event detection time at which the event is detected. For example, when the target pushes a button of the external optical receiver 1615, the external optical receiver 1615 may determine that the abnormal symptom occurs in the target.

In operation 1655, the external optical receiver 1615 may transmit the biometric data and information associated with the event detection time to the remote biometric data collector 1610. In operation 1660, the remote biometric data collector 1610 may transmit the biometric data and the information associated with the event detection time to the server 1605.

In operation 1665, the server 1605 may transmit alarm information to a terminal of a preset recipient address, in response to the information associated with the event detection time being received. The server 1605 may transmit the alarm information notifying that the abnormal symptom occurs in the target to a preset hospital/doctor, a public office/local government office, a family/acquaintance, or the target himself/herself, such that an appropriate measure for the abnormal symptom of the target is taken.

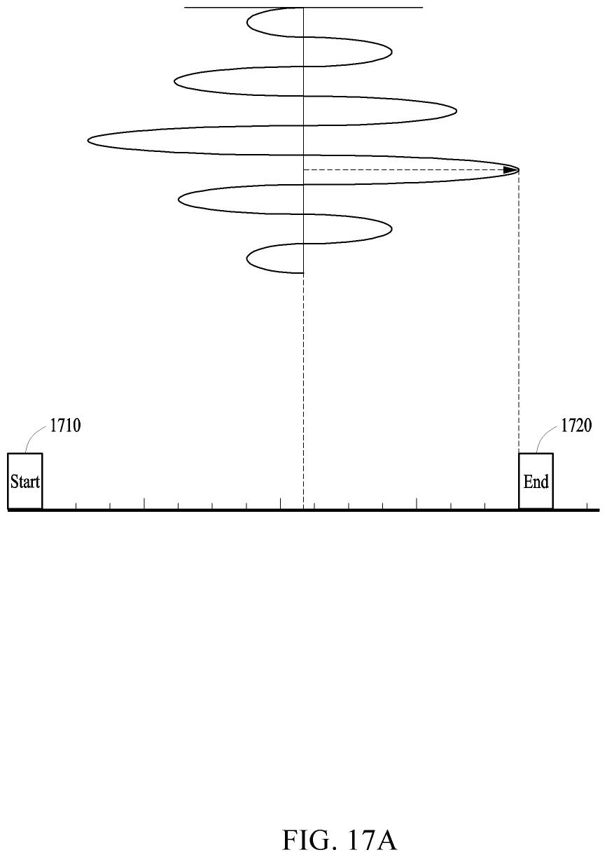

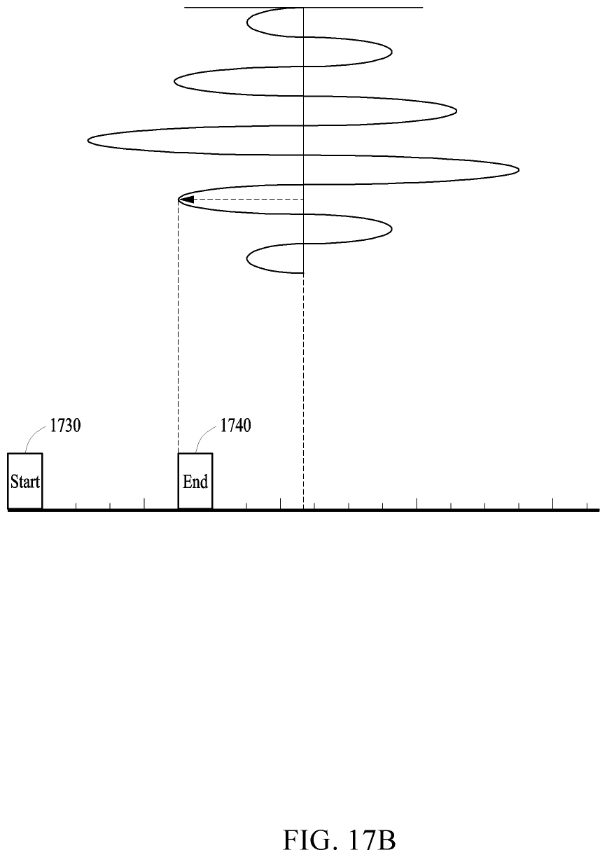

FIGS. 17A and 17B are diagrams illustrating examples of an optical signal according to an example embodiment.

Referring to FIGS. 17A and 17B, an optical signal transmitting device may determine a transmission time point of each of a start pulse and an end pulse corresponding to an optical signal, based on successive biometric data of a curved and decimal form. The optical signal transmitting device may encode the biometric data. The optical signal transmitting device may modulate the encoded biometric data into an optical signal. The optical signal may represent a data value of the biometric data based on a time interval between the start pulse and the end pulse.

Referring to FIGS. 17A and 17B, there are spaces between a start pulse and an end pulse with certain intervals therebetween. An optical signal receiving device may recognize a time interval between the start pulse and the end pulse based on the number of spaces therebetween. For example, there may be 1024 spaces that are divided by a certain interval.

According to an example embodiment, the optical signal transmitting device may determine a time interval between a time point at which a start pulse is transmitted and a time point at which an end pulse is transmitted, based on a data value of biometric data. In addition, the optical signal transmitting device may determine the time point at which the end pulse is transmitted based on a time interval between the start pulse and the end pulse.

Referring to FIG. 17A, to transmit a first optical signal to the optical signal receiving device, the optical signal transmitting device may transmit a start pulse 1710 corresponding to the first optical signal to the optical signal receiving device. The optical signal receiving device may start a time count, starting from a time point at which the optical signal receiving device receives the start pulse 1710 corresponding to the first optical signal. The optical signal transmitting device may determine a time interval between the start pulse 1710 and an end pulse 1720 based on a data value corresponding to the optical signal. The optical signal transmitting device may determine a transmission time point at which the end pulse 1720 is to be transmitted to the optical signal receiving device after a time point at which the start pulse 1710 is transmitted to the optical signal receiving device, based on the time interval between the start pulse 1710 and the end pulse 1720. The optical signal transmitting device may transmit the end pulse 1720 to the optical signal receiving device based on the determined transmission time point. The optical signal receiving device may end the time count at a time point at which the end pulse 1720 is received from the optical signal transmitting device. The optical signal receiving device may extract biometric data corresponding to the first optical signal based on a result of the time count.

Referring to FIG. 17B, to transmit a second optical signal to the optical signal receiving device, the optical signal transmitting device may transmit a start pulse 1730 corresponding to the second optical signal to the optical signal receiving device. The optical signal receiving device may start a time count, starting from a time point at which the start pulse 1730 corresponding to the second optical signal is received. The optical signal transmitting device may determine a time interval between the start pulse 1730 and an end pulse 1740 based on a data value corresponding to the optical signal. The optical signal transmitting device may determine a transmission time point at which the end pulse 1740 is to be transmitted to the optical signal receiving device after a time point at which the start pulse 1730 is transmitted to the optical signal receiving device, based on the time interval between the start pulse 1730 and the end pulse 1740. The optical signal transmitting device may transmit the end pulse 1740 to the optical signal receiving device based on the determined transmission time point. The optical signal receiving device may end the time count at a time point at which the end pulse 1740 is received from the optical signal transmitting device. The optical signal receiving device may extract biometric data corresponding to the second optical signal based on a result of the time count.

Comparing the example of FIG. 17A and the example of FIG. 17B, it is verified that the time interval between the start pulse 1710 and the end pulse 1720 corresponding to the first optical signal is greater than the time interval between the start pulse 1730 and the end pulse 1740 corresponding to the second optical signal. Based on this, it may be determined that a magnitude of the data value corresponding to the first optical signal is greater than a magnitude of the data value corresponding to the second optical signal. That is, when a time interval between a start pulse and an end pulse corresponding to an optical signal is greater than a time interval between a start pulse and an end pulse corresponding to another optical signal, a data value corresponding to the optical signal may be determined to be greater than a data value corresponding to the other optical signal. In contrast, when a time interval between a start pulse and an end pulse corresponding to an optical signal is less than a time interval between a start pulse and an end pulse corresponding to another optical signal, a data value corresponding to the optical signal may be determined to be less than a data value corresponding to the other optical signal.

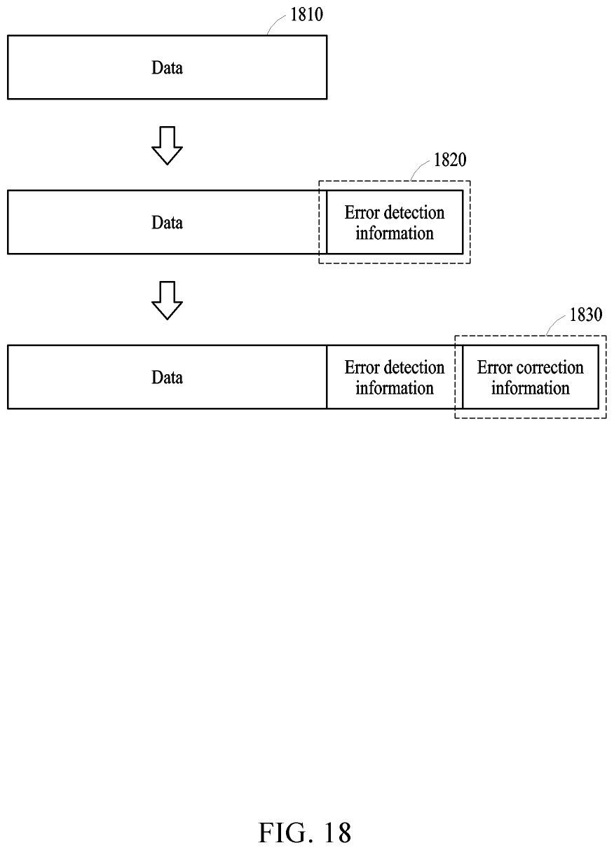

FIG. 18 is a diagram illustrating an example of data to which error detection information and error correction information are added according to an example embodiment.

Referring to FIG. 18, according to the first example embodiment, an optical signal transmitting device may collect biometric data 1810. To prevent occurrence of an error, the optical signal transmitting device may add at least one of error detection information 1820 and error correction information 1830 to the collected biometric data 1810.

The optical signal transmitting device may add the error detection information 1820 to the biometric data 1810 such that an optical signal receiving device receiving the biometric data 1810 determines an error of the biometric data 1810. The error detection information 1820 may be a parity bit, for example. The optical signal receiving device may determine whether there is an error in the received biometric data 1810 based on a parity bit corresponding to the error detection information 1820.

In addition, the optical signal transmitting device may add the error correction information 1830 to the biometric data 1810 such that the optical signal receiving device receiving the biometric data 1810 corrects the error of the biometric data 1810. The error correction information 1830 may be information for correcting an error, for example, Bose-Chaudhuri-Hocquenghem (BCH) codes. The optical signal receiving device may obtain information associated with the size of the biometric data 1810 and related information based on the BCH codes corresponding to the error correction information 1830, and correct the error of the biometric data 1810 when there is the error in the biometric data 1810 based on the obtained information.

According to examples, the optical signal transmitting device may add at least one of the error detection information 1820 and the error correction information 1830 to the biometric data 1810. Alternatively, at least one of the error detection information 1820 and the error correction information 1830 may be already added to biometric data received by the optical signal transmitting device.

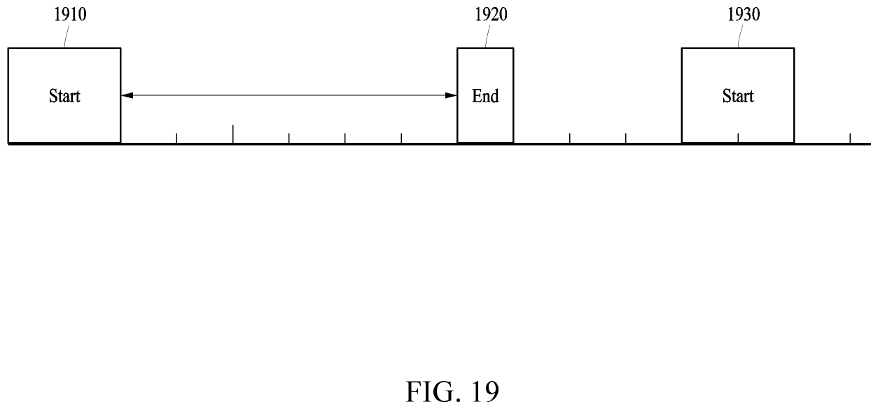

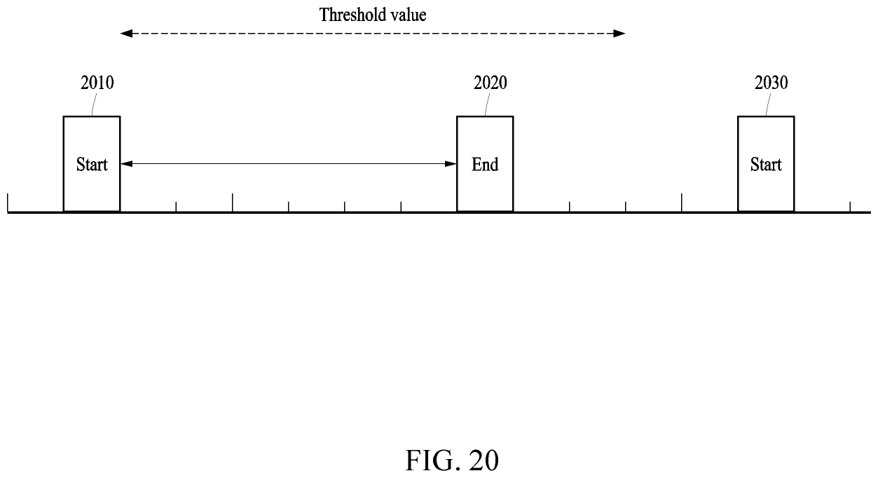

FIG. 19 is a diagram illustrating an example of an optical signal in which a start pulse and an end pulse have different widths according to the second example embodiment. Referring to FIG. 19, according to the second example embodiment, an optical signal transmitting device may transmit a start pulse 1910 corresponding to a first optical signal and an end pulse 1920 corresponding to the first optical signal. A time interval between the start pulse 1910 and the end pulse 1920 may correspond to six spaces, and thus an optical signal receiving device may extract, from the first optical signal, a data value corresponding to the six spaces corresponding to the time interval between the start pulse 1910 and the end pulse 1920 corresponding to the first optical signal. The optical signal receiving device may wait until receiving a start pulse corresponding to a subsequent optical signal after receiving the end pulse 1920 corresponding to the first optical signal. The optical signal receiving device may receive a start pulse 1930 corresponding to a second optical signal, and start a time count until receiving an end pulse corresponding to the second optical signal in response to the reception of the start pulse 1930.

The optical signal receiving device may recognize and identify the start pulses 1910 and 1930 and the end pulse 1920 based on a characteristic that the start pulses 1910 and 1930 and the end pulse 1920 have different widths. A pulse width used herein may represent a time interval in which a signal value is maintained or a time interval in which a logic level is maintained at 1. That is, the pulse width may indicate a time interval in which the optical signal transmitting device maintains outputting light.

When the optical signal receiving device receives the start pulse 1910 corresponding to the first optical signal, the optical signal receiving device may recognize a start of reception of the first optical signal, based on the characteristic that the start pulses 1910 and 1930 and the end pulse 1920 have different widths. The optical signal receiving device recognizing the start of the reception of the first optical signal may start a time count. When the optical signal receiving device receives the start pulse 1930 corresponding to the second optical signal while waiting to receive the end pulse 1920 corresponding to the first optical signal, the optical signal receiving device may suspend the time count corresponding to the first optical signal and start a time count corresponding to the second optical signal. The optical signal receiving device may identify the start pulses 1910 and 1930 and the end pulse 1920. Thus, even when an error occurs in a process of transmitting and receiving an optical signal, the optical signal receiving device may minimize an influence of the error on the processing of a subsequent optical signal to be received by the optical signal receiving device.

FIG. 20 is a diagram illustrating an example of an optical signal having a threshold value according to the third example embodiment.

Referring to FIG. 20, according to the third example embodiment, an optical signal transmitting device may set a threshold value for an optical signal and share the set threshold value with an optical signal receiving device. A threshold value for an optical signal used herein may be a maximum value of a time interval between a start pulse and an end pulse corresponding to the optical signal.

According to the example embodiment, the optical signal transmitting device may determine the threshold value and share the determined threshold value with the optical signal receiving device. Referring to FIG. 20, the optical signal receiving device may receive a start pulse 2010 corresponding to a first optical signal, and then receive an end pulse 2020 corresponding to the first optical signal. The optical signal receiving device may receive normally the first optical signal by receiving the end pulse 2020 corresponding to the first optical signal before reaching a time count corresponding to a threshold value after receiving the start pulse 2010 corresponding to the first optical signal. After receiving the end pulse 2020 corresponding to the first optical signal, the optical signal receiving device may receive a start pulse 2030 corresponding to a second optical signal. The optical signal receiving device may wait to receive an end pulse corresponding to the second optical signal in order to receive normally the second optical signal.

However, when an error occurs in receiving one pulse because respective widths or wavelengths of the start pulses 2020 and 2030 and the end pulse 2020 are the same and thus the optical signal receiving device fails to identify a start pulse and an end pulse from each other using only a pulse width or a wavelength, this one error may affect even the reception of a subsequent optical signal by the optical signal receiving device. For example, when, in the absence of a threshold value, the optical signal receiving device does not receive the end pulse 2020 corresponding to the first optical signal after receiving the start pulse 2010 corresponding to the first optical signal, the optical signal receiving device may extract erroneous biometric data from optical signals received from the optical signal transmitting device by recognizing the start pulse 2030 corresponding to the second optical signal as an end pulse corresponding to the first optical signal.

However, when, in the presence of a threshold value, the optical signal receiving device does not receive the end pulse 2020 corresponding to the first optical signal, the optical signal receiving device may determine that an error occurs in receiving the first optical signal based on the threshold value. Since the optical signal receiving device receives the start pulse 2030 corresponding to the second optical signal after a time interval corresponding to the threshold value elapses from a time point at which the start pulse 2010 corresponding to the first optical signal is received, the optical signal receiving device may recognize the start pulse 2030 corresponding to the second optical signal without an influence of an error. For example, the optical signal receiving device may start a time count after receiving the start pulse 2010 corresponding to the first optical signal. In this example, when the optical signal receiving device dos not receive the end pulse 2020 corresponding to the first optical signal within the time count corresponding to the threshold value, the optical signal receiving device may suspend the time count corresponding to the first optical signal and wait for the start pulse 2030 corresponding to the second optical signal.

According to another example embodiment, the threshold value may also be applied to the transmission of sub-biometric data. For example, the optical signal transmitting device may divide 16-bit biometric data into two sub-biometric data sets. The optical signal transmitting device may divide the 16-bit biometric data into 8-bit first sub-biometric data and 8-bit second sub-biometric data. The optical signal transmitting device may modulate the first sub-biometric data into a first optical signal and the second sub-biometric data into a second optical signal, and transmit the first optical signal and the second optical signal to the optical signal receiving device. When the optical signal receiving device successfully receives the first optical signal and obtains the first sub-biometric data from the first optical signal, the optical signal receiving device may wait to receive the second optical signal corresponding to the second sub-biometric data to be combined with the first sub-biometric data. However, when the optical signal receiving device does not receive the second optical signal even after a time corresponding to a preset threshold value elapses, the optical signal receiving device may recognize that an error occurs in optical signal communication. In such a case, the optical signal receiving device may discard the first sub-biometric data corresponding to the received first optical signal and wait for an optical signal corresponding to new biometric data.

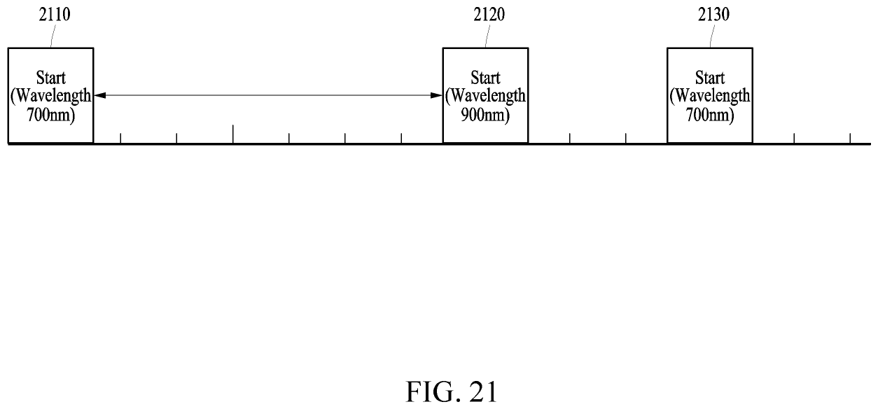

FIG. 21 is a diagram illustrating an example of an optical signal in which a start pulse and an end pulse have different wavelengths according to a fourth example embodiment.

Referring to FIG. 21, according to the fourth example embodiment, an optical signal may include a start pulse and an end pulse. Here, a wavelength of the start pulse may be different from a wavelength of the end pulse.

An optical signal transmitting device may transmit a start pulse 2110 corresponding to a first optical signal and an end pulse 2120 corresponding to the first optical signal to an optical signal receiving device. The optical signal receiving device may wait until receiving a start pulse corresponding to a subsequent optical signal after receiving the end pulse 2120 corresponding to the first optical signal. The optical signal receiving device may receive a start pulse 2130 corresponding to a second optical signal, and start a time count until receiving an end pulse corresponding to the second optical signal in response to the reception of the start pulse 2130.

A wavelength of the start pulses 2110 and 2130 and a wavelength of the end pulse 2120 may differ from each other. For example, the optical signal transmitting device may set the wavelength of the start pulses 2110 and 2130 to be 700 nm and the wavelength of the end pulse 2120 to be 900 nm. The optical signal receiving device may recognize and identify the start pulses 2110 and 2130 and the end pulse 2120 based on a characteristic that the wavelengths of the start pulses 2110 and 2130 and the end pulse 2120 are different.

When the start pulse 2110 corresponding to the first optical signal is received, the optical signal receiving device may recognize a start of reception of the first optical signal based on the characteristic that the wavelengths of the start pulses 2110 and 2130 and the end pulse 2120 are different. The optical signal receiving device recognizing the start of the reception of the first optical signal may start a time count. When the optical signal receiving device receives the start pulse 2130 corresponding to the second optical signal while waiting to receive the end pulse 2120 corresponding to the first optical signal, the optical signal receiving device may suspend the time count corresponding to the first optical signal and start a time count corresponding to the second optical signal. The optical signal receiving device may identify the start pulses 2110 and 2130 and the end pulse 2120 based on the respective wavelengths of the pulses 2110, 2120, and 2130. Thus, even when an error occurs in a process of transmitting and receiving an optical signal, the optical signal receiving device may minimize an influence of the error on the processing of a subsequent optical signal to be received by the optical signal receiving device.

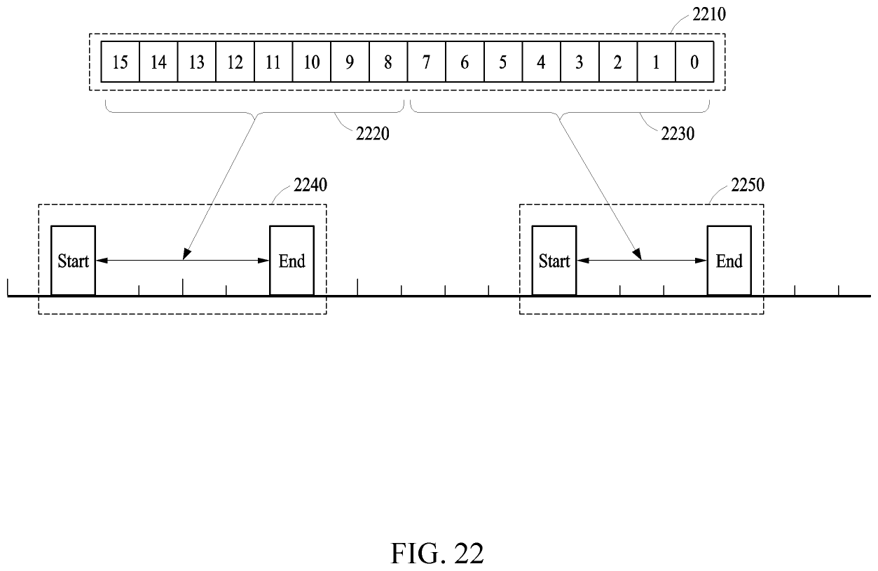

FIG. 22 is a diagram illustrating examples of optical signals generated based on sub-biometric data sets obtained by dividing biometric data according to a fifth example embodiment.

Referring to FIG. 22, according to the fifth example embodiment, an optical signal transmitting device may divide biometric data 2210 into a plurality of sub-biometric data sets 2220 and 2230, and generate optical signals 2240 and 2250 respectively corresponding to the sub-biometric data sets 2220 and 2230. For example, the biometric data 2210 may be divided into first sub-biometric data 2220 and second sub-biometric data 2230. In this example, the optical signal transmitting device may generate a first optical signal 2240 from the first sub-biometric data 2220 and a second optical signal 2250 from the second sub-biometric data 2230.

Each of the first optical signal 2240 and the second optical signal 2250 may include a start pulse and an end pulse. An optical signal receiving device may receive the first optical signal 2240 and the second optical signal 2250 by identifying them based on whether the start pulse and the end pulse are received. The optical signal receiving device may obtain the first sub-biometric data 2220 which is data corresponding to the first optical signal 2240 by demodulating the first optical signal 2240, and obtain the second sub-biometric data 2230 which is data corresponding to the second optical signal 2250 by demodulating the second optical signal 2250. The optical signal receiving device may obtain the biometric data 2210 by combining the first sub-biometric data 2220 and the second sub-biometric data 2230.

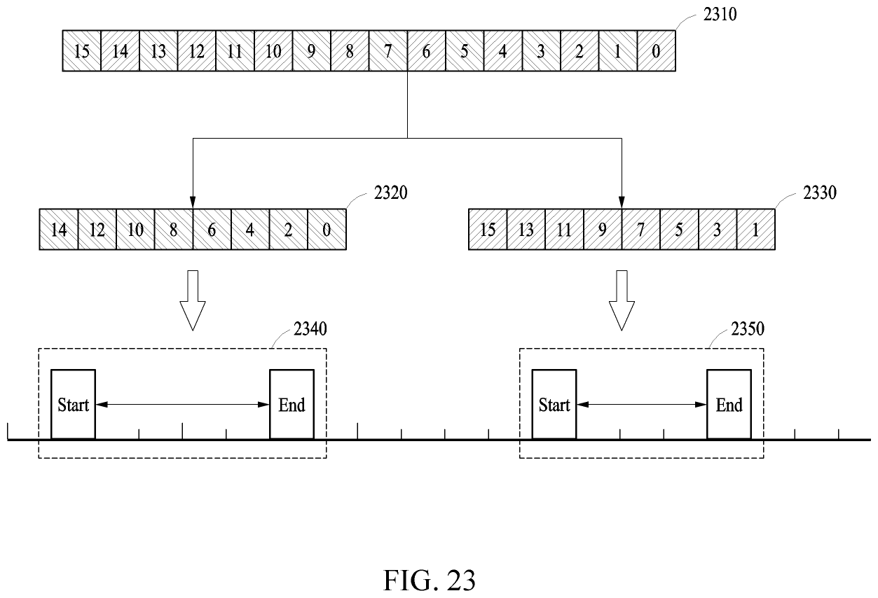

FIG. 23 is a diagram illustrating examples of optical signals generated as biometric data is divided into even bits and odd bits according to a sixth example embodiment.

Referring to FIG. 23, according to the sixth example embodiment, an optical signal transmitting device may divide biometric data 2310 into first sub-biometric data 2320 and second sub-biometric data 2330. The optical signal transmitting device may divide the biometric data 2310 into even bits and odd bits and thereby divide the biometric data 2310 into the first sub-biometric data 2320 including the even bits and the second sub-biometric data 2330 including the odd bits. The optical signal transmitting device may generate a first optical signal 2340 corresponding to the first sub-biometric data 2320 and a second optical signal 2350 corresponding to the second sub-biometric data 2330.

The optical signal transmitting device may transmit the first optical signal 2340 and the second optical signal 2350 to an optical signal receiving device. The optical signal receiving device may receive the first optical signal 2340 and the second optical signal 2350 based on a start pulse and an end pulse included in each of the first optical signal 2340 and the second optical signal 2350.

The optical signal receiving device may obtain the first sub-biometric data 2320 corresponding to the first optical signal 2340 by demodulating the first optical signal 2340, and obtain the second sub-biometric data 2330 corresponding to the second optical signal 2350 by demodulating the second optical signal 2350. The optical signal receiving device may obtain the biometric data 2310 transmitted from the optical signal transmitting device by combining the first sub-biometric data 2320 and the second sub-biometric data 2330.

A lower bit may be more vulnerable to a transmission and reception error that may occur when the optical signal transmitting device transmits an optical signal or the optical signal receiving device receives an optical signal. In addition, in the case of sub-biometric data corresponding to an upper bit, when a transmission and reception error occurs, a difference between the biometric data 2310 and biometric data obtained by the optical signal receiving device through demodulation may increase. A method through which the optical signal transmitting device divides the biometric data 2310 into a plurality of sub-biometric data sets and modulates the biometric data 2310 into an optical signal, and then transmits the optical signal to the optical signal receiving device may make the upper bit vulnerable to an error. Thus, according to the example embodiment, the optical signal transmitting device may divide the biometric data 2310 into even bits and odd bits, and divide the biometric data 2310 into the first sub-biometric data 2320 including the even bits and the second sub-biometric data 2330 including the odd bits. The optical signal transmitting device may generate the first optical signal 2340 corresponding to the first sub-biometric data 2320, and the second optical 2350 corresponding to the second sub-biometric data 2330. Thus, the optical signal transmitting device may minimize a transmission and reception error that may occur due to the division of the biometric data 2310. The division of the biometric data 2310 by the optical signal transmitting device is not limited to the foregoing example. According to examples, the optical signal transmitting device may divide the biometric data 2310 into three or more sub-biometric biometric data sets, and a method for the division is not limited to the foregoing example.

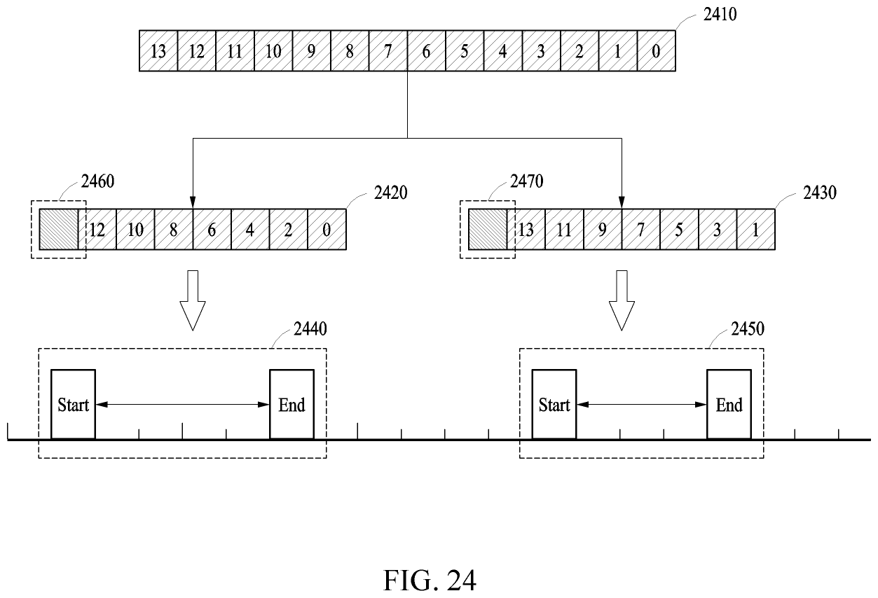

FIG. 24 is a diagram illustrating examples of optical signals each including an identifier bit according to a seventh example embodiment.

Referring to FIG. 24, according to the seventh example embodiment, an optical signal transmitting device may divide biometric data 2410 into first sub-biometric data 2420 and second sub-biometric data 2430. The optical signal transmitting device may generate a first optical signal 2440 based on the first sub-biometric data 2420 and transmit the first optical signal 2440 to an optical signal receiving device. In addition, the optical signal transmitting device may generate a second optical signal 2450 based on the second sub-biometric data 2430 and transmit the second optical signal 2450 to the optical signal receiving device.

The first sub-biometric data 2420 and the second sub-biometric data 2430 may include identifier bits 2460 and 2470, respectively, that indicate which ones of even bits and odd bits of the biometric data 2410 are included in the first sub-biometric data 2420 and the second sub-biometric data 2430. The first sub-biometric data 2420 may include the even bits of the biometric data 2410. In addition, the first sub-biometric data 2420 may include a first identifier bit 2460 indicating that the first sub-biometric data 2420 includes the even bits of the biometric data 2410. The second sub-biometric data 2430 may include the odd bits of the biometric data 2410. In addition, the second sub-biometric data 2430 may include a second identifier bit 2470 indicating that the second sub-biometric data 2430 includes the odd bits of the biometric data 2410.

According to another example embodiment, each of the first sub-biometric data 2420 and the second sub-biometric data 2430 may include an identifier bit to identify that they include different bits, regardless of whether they include even bits or odd bits. For example, the first sub-biometric data 2420 may include a data value of first bits, and further include a first identifier bit 2460 indicating that the first sub-biometric data 2420 includes the data value of the first bits. The second sub-biometric data 2430 may include a data value of second bits distinguished from the first bits, and further include a second identifier bit 2470 indicating that the second sub-biometric data 2430 includes the data value of the second bits.

The optical signal receiving device may receive the first optical signal 2440 and the second optical signal 2450. The optical signal receiving device may obtain the first sub-biometric data 2420 based on the first optical signal 2440, and obtain the second sub-biometric data 2430 based on the second optical signal 2450. The optical signal receiving device may combine the first sub-biometric data 2420 and the second sub-biometric data 2430 based on the first identifier bit 2460 included in the first sub-biometric data 2420 and the second identifier bit 2470 included in the second sub-biometric data 2430.

According to another example embodiment, the optical signal receiving device may receive the first optical signal 2440, and obtain the first sub-biometric data 2420 and the first identifier bit 2460 included in the first sub-biometric data 2420 from the first optical signal 2440. Afterward, when the optical signal receiving device receives an optical signal, but sub-biometric data and an identifier bit obtained from the received optical signal are not associated with the first identifier 2460, the optical signal receiving device may determine that there is an error in receiving sub-biometric data to be combined with the first sub-biometric data 2420. In such a case, the optical signal receiving device may determine not to receive the biometric data 2410 corresponding to the first sub-biometric data 2420, and wait to receive a subsequent optical signal.

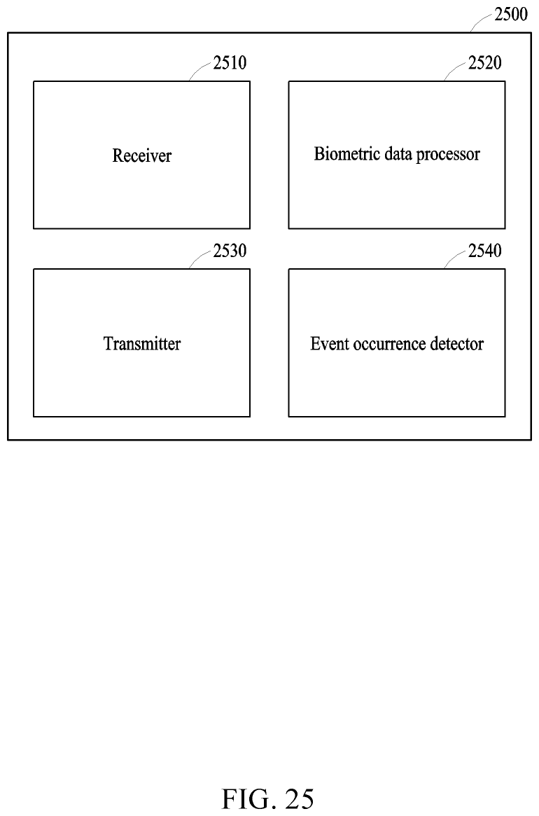

FIG. 25 is a diagram illustrating an example of a configuration of an external optical receiver according to an example embodiment.

Referring to FIG. 25, an external optical receiver 2500 may include a receiver 2510, a biometric data processor 2520, a transmitter 2530, and an event occurrence detector 2540. The external optical receiver 2500 described herein with reference to FIG. 25 may correspond to an external optical receiver and an optical signal receiving device described herein.

According to an example embodiment, functions and operations of the receiver 2510, the biometric data processor 2520, the transmitter 2530, and the event occurrence detector 2540 may be performed by one or more processors.

The receiver 2510 may receive an optical signal including biometric data from an implantable device. The biometric data processor 2520 may detect the biometric data from the optical signal. The transmitter 2530 may transmit the biometric data to a remote biometric data collector. The event occurrence detector 2540 may determine whether a preset event occurs to detect whether an abnormal symptom occurs in a target from which the biometric data is collected.

According to an example embodiment, it is possible to measure and monitor biometric data in real time.

According to an example embodiment, increasing the life of a battery of an implantable device may minimize a patient's inconvenience in having to get a surgical operation to replace the battery.

According to an example embodiment, using a communication method that is harmless to a human body, it is possible to improve safety.

According to an example embodiment, measuring and transmitting biometric data in real time may improve diagnostic accuracy.

According to an example embodiment, when a patient senses an abnormal symptom himself or herself, recording a time at which the abnormal symptom occurs may improve diagnostic accuracy.

According to an example embodiment, allowing an alarm to be automatically set off in case of emergency may facilitate a quick response or measure, thereby improving the safety of a patient.

Although example embodiments are described mainly with an example of biometric data monitoring applied to a human or human body, the scope of examples is not limited thereto. For example, the example embodiments may also be applied to biometric data monitoring applied to an animal.

The units described herein may be implemented using hardware components and software components. For example, the hardware components may include microphones, amplifiers, band-pass filters, audio to digital convertors, non-transitory computer memory and processing devices. A processing device may be implemented using one or more general-purpose or special purpose computers, such as, for example, a processor, a controller and an arithmetic logic unit (ALU), a digital signal processor, a microcomputer, a field programmable gate array (FPGA), a programmable logic unit (PLU), a microprocessor or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is used as singular; however, one skilled in the art will appreciated that a processing device may include multiple processing elements and multiple types of processing elements. For example, a processing device may include multiple processors or a processor and a controller. In addition, different processing configurations are possible, such as a parallel processors.

The software may include a computer program, a piece of code, an instruction, or some combination thereof, to independently or collectively instruct or configure the processing device to operate as desired. Software and data may be embodied permanently or temporarily in any type of machine, component, physical or virtual equipment, computer storage medium or device, or in a propagated signal wave capable of providing instructions or data to or being interpreted by the processing device. The software also may be distributed over network coupled computer systems so that the software is stored and executed in a distributed fashion. The software and data may be stored by one or more non-transitory computer readable recording mediums. The non-transitory computer readable recording medium may include any data storage device that can store data which can be thereafter read by a computer system or processing device.

The methods according to the above-described example embodiments may be recorded in non-transitory computer-readable media including program instructions to implement various operations of the above-described example embodiments. The media may also include, alone or in combination with the program instructions, data files, data structures, and the like. The program instructions recorded on the media may be those specially designed and constructed for the purposes of example embodiments, or they may be of the kind well-known and available to those having skill in the computer software arts. Examples of non-transitory computer-readable media include magnetic media such as hard disks, floppy disks, and magnetic tape; optical media such as CD-ROM discs, DVDs, and/or Blue-ray discs; magneto-optical media such as optical discs; and hardware devices that are specially configured to store and perform program instructions, such as read-only memory (ROM), random access memory (RAM), flash memory (e.g., USB flash drives, memory cards, memory sticks, etc.), and the like. Examples of program instructions include both machine code, such as produced by a compiler, and files containing higher level code that may be executed by the computer using an interpreter. The above-described devices may be configured to act as one or more software modules in order to perform the operations of the above-described example embodiments, or vice versa.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner and/or replaced or supplemented by other components or their equivalents.

Therefore, the scope of the disclosure is defined not by the detailed description, but by the claims and their equivalents, and all variations within the scope of the claims and their equivalents are to be construed as being included in the disclosure.

What is claimed is:

1. An optical signal communication method performed by an optical signal transmitting device, the optical signal communication method comprising:
receiving current input data to be modulated into an optical signal;
dividing the received current input data into a plurality of sub-data sets comprising first sub-data and second sub-data;
modulating the current input data into an optical signal comprising a start pulse, an intermediate pulse, and an end pulse based on the sub-data sets; and
transmitting the optical signal to an optical signal receiving device,
wherein, when the optical signal receiving device does not receive at least one of the intermediate pulse and the end pulse from the optical signal transmitting device after the optical signal receiving device receives the start pulse and reaches a time count corresponding to a threshold value, the optical signal receiving device is configured to determine not to demodulate the received optical signal.

2. The optical signal communication method of claim 1, wherein the first sub-data comprises an upper bit of the current input data, and the second sub-data comprises a lower bit of the current input data,
a bit number of the first sub-data is less than a bit number of the second sub-data,
an interval between the start pulse and the intermediate pulse is determined based on a data value of the first sub-data, and
an interval between the intermediate pulse and the end pulse is determined based on a data value of the second sub-data.

3. An optical signal communication method performed by an optical signal transmitting device, the optical signal communication method comprising:
receiving current input data to be modulated into an optical signal; and
modulating the current input data into an optical signal and transmitting the optical signal when a difference in value between the current input data and previous input data is greater than a threshold value, and not transmitting the current input data when the difference in value between the current input data and the previous input data is less than or equal to the threshold value,
wherein the transmitting the optical signal comprises transmiting the optical signal to an optical signal receiving device based on a preset transmission period shared between the optical signal transmitting device and the optical signal receiving device.

4. The optical signal communication method of claim 3, wherein the optical signal receiving device is configured to:
receive a current optical signal from the optical signal transmitting device based on the preset transmission period; and
when the optical signal receiving device does not receive the current optical signal from the optical signal transmitting device based on the preset transmission period, determine the current input data based on a most recently received optical signal.

5. The optical signal communication method of claim 3, wherein the modulating comprises:
when the difference in value between the current input data and the previous input data is greater than the threshold value, modulating the current input data into an optical signal comprising a start pulse, an intermediate pulse, and an end pulse based on divided sub-data sets.

6. A biometric data monitoring system, comprising:
an implantable device configured to be inserted in a body of a target from which biometric data is to be collected, collect the biometric data, modulate the biometric data into an optical signal of a near-infrared light wavelength, and transmit the optical signal to an external optical receiver outside the body;
the external optical receiver configured to detect the biometric data from the received optical signal outside the body of the target and transmit the detected biometric data to a remote biometric data collector; and
the remote biometric data collector configured to transmit the received biometric data to a server.

7. The biometric data monitoring system of claim 6, wherein the implantable device is configured to:
receive current input data to be modulated into an optical signal;
divide the received current input data into a plurality of sub-data sets comprising first sub-data and second sub-data;
modulate the current input data into an optical signal comprising a start pulse, an intermediate pulse, and an end pulse based on the sub-data sets; and
transmit the optical signal to the external optical receiver.

8. The biometric data monitoring system of claim 7, wherein the first sub-data comprises an upper bit of the current input data, and the second sub-data comprises a lower bit of the current input data,
a bit number of the first sub-data is less than a bit number of the second sub-data,
an interval between the start pulse and the intermediate pulse is determined based on a data value of the first sub-data, and
an interval between the intermediate pulse and the end pulse is determined based on a data value of the second sub-data.

9. The biometric data monitoring system of claim 7, wherein the external optical receiver is configured to:
when the external optical receiver does not receive at least one of the intermediate pulse and the end pulse from the implantable device after the external optical receiver receives the start pulse and reaches a time count corresponding to a threshold value, determine not to demodulate the received optical signal.

10. The biometric data monitoring system of claim 6, wherein the implantable device is configured to:
receive current input data to be modulated into an optical signal;
modulate the current input data into the optical signal when a difference in value between the current input data and previous input data is greater than a threshold value, and not transmit the current input data when the difference in value between the current input data and the previous input data is less than or equal to the threshold value; and
transmit the optical signal to the external optical receiver based on a preset transmission period shared between the implantable device and the external optical receiver.

11. The biometric data monitoring system of claim 10, wherein the external optical receiver is configured to:
receive a current optical signal from the implantable device based on the preset transmission period; and
when the external optical receiver does not receive the current optical signal from the implantable device based on the preset transmission period, determine the current input data based on a most recently received optical signal.

12. The biometric data monitoring system of claim 6, wherein, when a preset event is detected, the external optical receiver is configured to determine that an abnormal symptom occurs in the target, and record an event detection time at which the event is detected, and
the remote biometric data collector is configured to transmit, to the server, the received biometric data and information associated with the event detection time.

13. The biometric data monitoring system of claim 12, wherein the server is configured to:
when the server receives the information associated with the event detection time from the remote biometric data collector, transmit alarm information notifying that the abnormal symptom occurs in the target to a terminal of a preset recipient.

14. The biometric data monitoring system of claim 12, wherein the preset event is detected in at least one of cases where the external optical receiver receives a user button input to a preset button, and where a preset application that detects an abnormal symptom of the target is executed in the remote biometric data collector.

15. The biometric data monitoring system of claim 12, wherein the preset event is detected in at least one of cases where a preset switch of the remote biometric data collector is activated, and where the remote biometric data collector is shaked at a certain or higher intensity.

16. The biometric data monitoring system of claim 12, wherein the remote biometric data collector is configured to:
transmit all collected biometric data sets, compress all the collected biometric data sets and transmit a result of the compressing, or transmit only biometric data corresponding to the information associated with the event detection time, based on at least one of a numerical value of a degree of severity of the abnormal symptom, a communication amount required for transmitting the biometric data, and a residual battery amount.
